(19) United States
(12) Patent Application Publication
(10) Pub. No.: US 2023/0203187 A1
(43) Pub. Date: Jun. 29, 2023

(54) Methods and Compositions to Treat Vascular Leak

(71) Applicant: Yale University, New Haven, CT (US)

(72) Inventors: Michael Simons, New Haven, CT (US); Federico Corti, New Haven, CT (US)

(21) Appl. No.: 17/999,639

(22) PCT Filed: May 21, 2021
(86) PCT No.: PCT/US2021/033759

§ 371 Date: Nov. 22, 2022

Related U.S. Application Data
(60) Provisional application No. 63/029,062, filed on May 22, 2020.

(51) Int. Cl. C07K 16/28; A61K 38/10; A61K 38/17

(52) U.S. Cl. CPC C07K 16/2896; A61K 38/10; A61K 38/1709; C07K 2317/76; A61K 39/00

(57) ABSTRACT

Provided are anti-syndecan-2 antibodies and compositions comprising said antibody and methods useful for treating acute respiratory distress syndrome, including coronavirus disease 2019-induced acute respiratory distress syndrome, in a subject in need thereof. The method comprises administering to the subject an effective amount to treat the acute respiratory distress syndrome of an syndecan-2 disrupting agent. The syndecan-2 disrupting agents may include at least one of the anti-syndecan-2 antibodies and/or at least one syndecan-2 disrupting peptide.

### CROSS-REFERENCE TO RELATED APPLICATIONS

This application claims to priority under 35 U.S.C. § 119(e) to U.S. Provisional Patent Application No. 63/029,062 filed May 22, 2020, which is hereby incorporated herein by reference in its entirety.

### STATEMENT REGARDING FEDERALLY SPONSORED RESEARCH OR DEVELOPMENT

This invention was made with government support under HL062289 awarded by National Institutes of Health. The government has certain rights in the invention.

### SEQUENCE LISTING

The ASCII text file named “047162-7286WO1 Sequence Listing” created on May 21, 2021, comprising 96 Kbytes, is hereby incorporated by reference in its entirety.

### BACKGROUND OF THE INVENTION

Vascular leakage associated with inflammation and tissue injury is a factor in a wide variety of pathologies. See Lange et al., Nature Reviews Neurology, published online Jul. 1, 2016. Syndecan-2 (SDC2) plays a significant role in regulating vascular permeability. SDC2 knock-out mice exhibit reduced vascular leakage after stimulation of vascular endothelial growth factor (VEGF) signaling. There is a need in the art for methods and compositions that inhibit SDC2 signaling, thereby treating vascular leakage, including acute respiratory distress syndrome (ARDS). The present disclosure addresses this need.

### SUMMARY OF THE INVENTION

In one aspect, the invention provides an anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

In various embodiments, the first amino acid sequence is selected from the group consisting of SEQ ID NOs: 1, 3, 5, 7, and 9 and the second amino acid sequence is selected from the group consisting of SEQ ID NOs: 2, 4, 6, 8, and 10.

In various embodiments, the antibody is an IgA, an IgD, an IgE, an IgG, or an IgM.

In various embodiments, the antibody further comprises a heavy chain constant region and a light chain constant region, wherein the heavy chain constant region comprises a third amino acid sequence having at least 90.5% sequence identity to SEQ ID NO: 36 and the light chain constant region comprises a fourth amino acid sequence having at least 91.5% sequence identity to SEQ ID NO: 37.

In various embodiments, the third amino acid sequence is SEQ ID NO: 36 and the fourth amino acid sequence is SEQ ID NO: 37.

In various embodiments, the antibody comprises a fifth amino acid sequence comprising SEQ ID NO: 38 is fused to the heavy chain variable region and a sixth amino acid sequence comprising SEQ ID NO: 39 is fused to the light chain variable region.

In various embodiments, the first amino acid sequence and the second amino acid sequence are respectively selected from the group consisting of SEQ ID NOs: 1 and 2, 3 and 4, 5 and 6, 7 and 8, and 9 and 10.

In another aspect, the invention provides a method of treating a syndecan-2 associated disease in a subject in need thereof, the method comprising administering to the subject an effective amount of an anti-syndecan-2 antibody to treat the ARDS, the anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

In various embodiments, the syndecan-2 associated disease is acute respiratory distress syndrome (ARDS), stroke, a neurological disease in which the blood brain barrier (BBB) is altered or disrupted, neovascular eye disease, a cardiovascular disease with a component of vascular hyperpermeability.

In various embodiments, the syndecan-2 associated disease is coronavirus disease 2019-induced ARDS (COVID-19-induced ARDS).

In various embodiments, the anti-syndecan-2 antibody is conjugated to a heterologous peptide.

In various embodiments, the subject is a mammal.

In various embodiments, the subject is a human.

In various embodiments, the anti-syndecan-2 antibody is administered in a dosage form further comprising at least one pharmaceutically acceptable carrier.

In another aspect, the invention provides a method of treating acute respiratory distress syndrome (ARDS) in a subject in need thereof, the method comprising administering to the subject an effective amount of a syndecan-2 disrupting peptide comprising an amino acid sequence set forth in SEQ ID NOs: 31, 32, or 33.

In various embodiments, the ARDS is coronavirus disease 2019-induced ARDS (COVID-19-induced ARDS).

In various embodiments, the syndecan-2 disrupting peptide is conjugated to a heterologous peptide.

In various embodiments, the heterologous peptide is selected from the group consisting of a cell penetrating peptide, a secretion signal peptide, or a stability enhancing domain.

In various embodiments, is a mammal.

In various embodiments, the subject is a human.

In various embodiments, the syndecan-2 disrupting peptide is administered in a dosage form further comprising at least one pharmaceutically acceptable carrier.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A: Scheme of full-length Sdc2 and relative position of DEP1-binding region inside the D2 domain. FIG. 1B: An anti-mouse Sdc2 antibody (Ab3) was raised against the mouse DEP1-binding motif in rabbit. FIG. 1C: Clones indicated in shading have been selected for production of human IgG1 antibodies in CHO cells. Four clones against AG3 and one clone against AG1.

FIG. 2A: Specificity of the newly developed Ab3 was tested with direct ELISA. Briefly, plates were coated either with mouse Sdc2 or mouse Sdc4 (amount in x-axis) and antibody binding was evaluated by colorimetric reaction (O.D, optical-density in y-axis). Ab3 display high specificity for mouse Sdc2 compared to mouse Sdc4. FIG. 2B: Preincubation of mouse brain endothelial cells (bEnd3 cells) with Ab3 leads to selective downregulation of VEGFR2 Y951 phosphorylation site while Y1175 is normally activated. FIGS. 2C and 2D: Ab3 selectively inhibits VEGFA165-induced permeability in vitro but does not affect endothelial cell proliferation.

FIGS. 3A-3B: Ab3 inhibits VEGF-induced permeability in vivo (Miles Assay). Ab3 was administrated I.V. with indicated dose (blood concentration in parenthesis) and left circulating for 2 hrs. Mice were then injected with 1% Evans Blue following by skin-permeability induction with VEGF or PBS as control. FIGS. 3C-3D: Ab3 effect in stroke was evaluated in a mouse model of Middle Cerebral Artery (MCA) permanent occlusion followed by TTC staining. Decreased infarct size (35% decrease) is observed in Ab3-treated animals versus control animals (IgG).

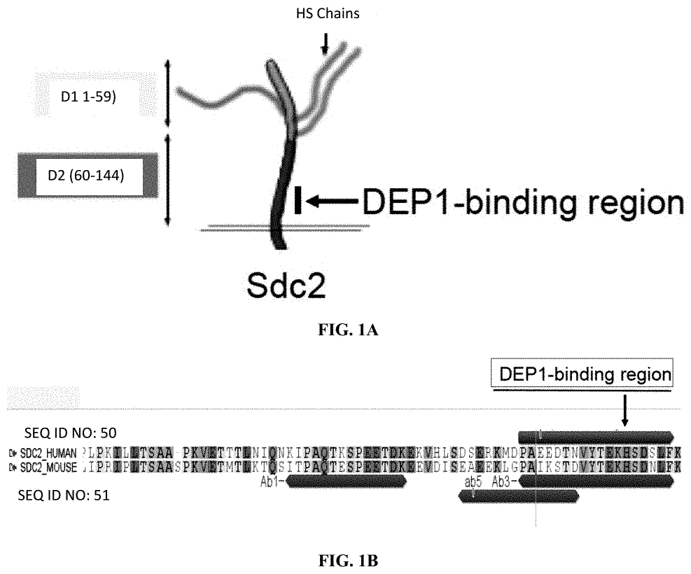

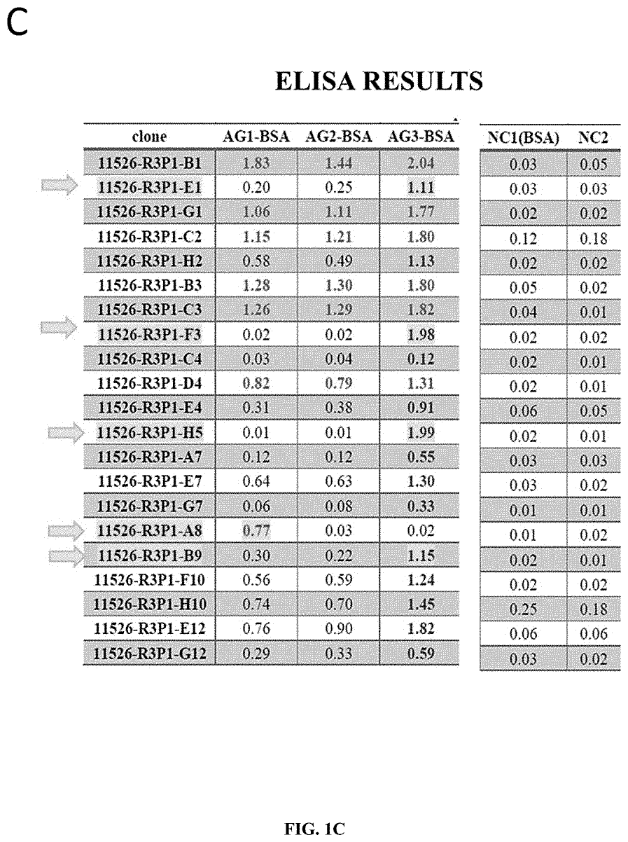

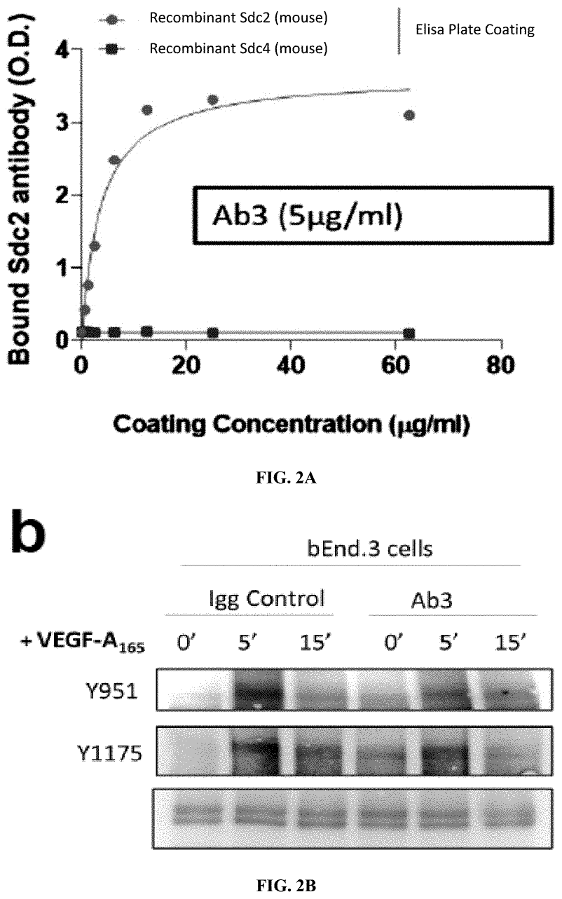

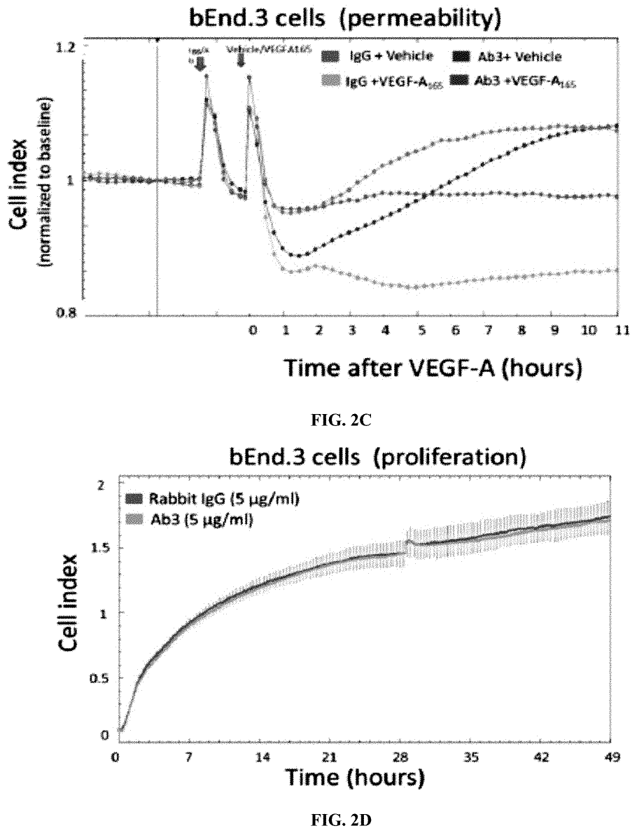

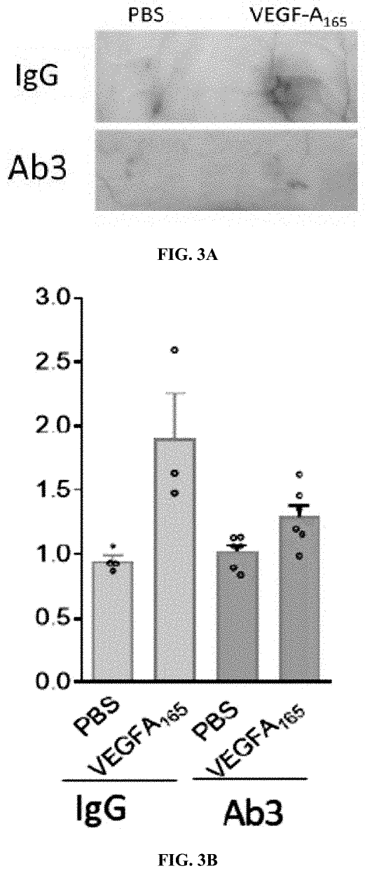

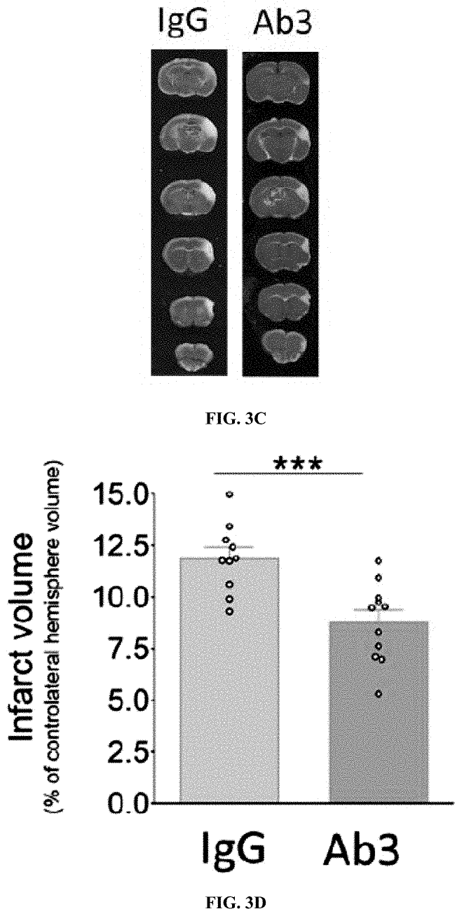

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

The term “antibody,” as used herein, refers to an immunoglobulin molecule which specifically binds with an antigen. Antibodies can be intact immunoglobulins derived from natural sources or from recombinant sources and can also be immunoreactive portions or immunoreactive fragments of intact immunoglobulins.

The term “antibody fragment,” as used herein, refers to an immunoreactive portion of an intact antibody.

In this regard, whether the antibody is an antibody fragment or an intact immunoglobulin, an antibody comprises variable regions, including a heavy chain variable region and a light chain variable region, which determine antigenicity. Antibodies are typically tetramers of immunoglobulin molecules. Antibodies may exist in a variety of forms including, for example, polyclonal antibodies, monoclonal antibodies, Fv, Fab, and F(ab)2, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). Examples of antibody fragments include, but are not limited to, Fab, Fab′, F(ab′)2, and Fv fragments, linear antibodies, scFv antibodies, and multispecific antibodies formed from antibody fragments.

The term “anti-syndecan-2 antibody” refers to an antibody that specifically binds to syndecan-2 under physiological conditions.

An “antibody heavy chain,” as used herein, refers to the larger of the two types of polypeptide chains present in all antibody molecules in their naturally occurring conformations. The antibody heavy chain comprises the heavy chain variable region and the heavy chain constant region.

An “antibody light chain,” as used herein, refers to the smaller of the two types of polypeptide chains present in all antibody molecules in their naturally occurring conformations. α and β light chains refer to the two major antibody light chain isotypes. The antibody light chain comprises the light chain variable region and the light chain constant region. Together, the light chain variable region(s) and the heavy chain variable region(s) of an antibody determine the antigenicity of the antibody.

By the term “synthetic antibody” as used herein, is meant an antibody which is generated using recombinant DNA technology, such as, for example, an antibody expressed by a CHO cells as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “coronavirus disease 2019” (COVID-19), as used herein, refers to the disease caused initially by infection of a subject with the novel 2019 coronavirus. The novel 2019 coronavirus is also known as severe acute respiratory syndrome coronavirus 2 (SARS-CoV-2). COVID-19, while caused initially by the infection with the SARS-CoV-2, is characterized in that it triggers a severe immune response in a subpopulation of individuals. The immune response to the SARS-CoV-2 virus and to the cells infected therefrom, in combination with the damage to the cells of the lung caused by the SARS-CoV-2 virus itself, can lead to acute respiratory distress syndrome in a subset of patients. COVID-19 can thereby require intubation, mechanical ventilation, and/or the use of a heart and lung bypass machine in a further subset of patients.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

As used herein, the term “heterologous peptide” refers to any peptide, polypeptide or protein whose sequence is selected in such a way that the product of the fusion of this sequence has a sequence different from the wild-type sequence flanking the peptide to which it is fused.

As used herein, “syndecan-2” or “SDC2” may refer to the protein having the sequence:

Human
SEQ ID NO: 34
MRRAWILLTLGLVACVSAESRAELTSDKDMYLDNSSIEE
ASGVYPIDDDDYASASGSGADEDVESPELTTSRPLPKILL
TSAAPKVETTTLNIQNKIPAQTKSPEETDKEKVHLSDSER
KMDPAEEDTNVYTEKHSDSLFKRTEVLAAVIAGGVIGFL
FAIFLILLLVYRMRKKDEGSYDLGERKPSSAAYQKAPTK
EFYA
Mouse
SEQ ID NO: 35
MQRAWILLTLGLMACVSAETRTELTSDKDMYLDNSSIE
EASGVYPIDDDDYSSASGSGADEDIESPVLTTSQLIPRIPL
TSAASPKVETMTLKTQSITPAQTESPEETDKEEVDISEAE
EKLGPAIKSTDVYTEKHSDNLFKRTEVLAAVIAGGVIGF
LFAIFLILLLVYRMRKKDEGSYDLGERKPSSAAYQKAPT
KEFYA

for the human and mouse homologs, or the gene encoding this protein.

A “syndecan-2 disrupting agent” as used herein, means an agent that interferes with the action of syndecan-2, as a non-limiting example by degrading the syndecan-2 protein or interfering with its production, or by preventing the binding of syndecan-2 to a ligand, as a non-limiting example, by blocking the binding site. In various embodiments, the syndecan-2 disrupting agent prevents Dep1-syndecan-2 interaction. As a non-limiting example, a syndecan-2 disrupting agent may include dominant negative forms of syndecan-2, e.g. the extracellular domain of syndecan-2 that binds Dep1. Without wishing to be bound to a particular theory, a dominant negative can bind Dep1 thereby reducing or preventing the binding of Dep1 to functional syndecan-2, which in turn thereby reduces or prevents the signaling of functional syndecan-2. As another non-limiting example, a syndecan-2 disrupting agent may include an antibody against syndecan-2 (an anti-syndecan-2 antibody). Without wishing to be bound by a particular theory, the anti-syndecan-2 antibody could bind to the Dep1 binding site on syndecan-2 thereby disrupting the binding of Dep1 and reducing or preventing syndecan-2 activation and signaling.

As used herein, the term “syndecan-2 extracellular domain” refers to a peptide having the sequence of the extracellular domain of syndecan-2 and including its associated heparan sulfate chains, either isolated or linked to a heterologous peptide. The amino acid sequence of the extracellular domain of human syndecan-2 is SEQ ID NO: 33:

```
                10 20 30 40
MYLDNSSIEE ASGVYPIDDD DYASASGSGA DEDVESPELT
                50 60 70 80
TSRPLPKILL TSAAPKVETT TLNIQNKIPA QTKSPEETDK
                90 100
EKVHLSDSERKMDPAEEDTN
              110 120 130
VYTEKHSDSL FKRTEVLAAV IAGGVIGFLF AIFLILL
```

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human or a non-human mammal. Non-human mammals include, for example, livestock and pets, such as ovine, bovine, porcine, canine, feline, and murine mammals. Preferably, the subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Anti-Syndecan-2 Antibodies

The invention is based in part on the discovery of one or more anti-syndecan-2 antibodies that modulate the signaling of syndecan-2, including by inhibiting syndecan-2 signaling, activating syndecan-2 signaling (i.e. in the absence of an agonist), or by potentiating syndecan-2 signaling in the presence of an agonist. In some embodiments, the antibody may bind to the extracellular region of syndecan-2, and more specifically, to the region of the extracellular domain of syndecan-2 to which Dep-1 binds. The anti-syndecan-2 antibody may thereby inhibit or activate syndecan-2 signaling. The invention is also based in part on the discovery that modulation of syndecan-2 can be used to treat acute respiratory distress syndrome (ARDS). A syndecan-2 disrupting agent, including a syndecan-2 disrupting peptide and an anti-syndecan-2 antibody, can prevent the vascular leak and pulmonary edema associated with ARDS.

The present invention provides an anti-syndecan-2 antibody, compositions comprising an anti-syndecan-2 antibody, and methods for using the same. The present invention also provides method of using a syndecan-2 disrupting agent, which may include one or more of the anti-syndecan-2 antibodies and/or one or more of a syndecan-2 disrupting peptide.

Regarding the anti-syndecan-2 antibody and compositions comprising said antibody, in various embodiments, an anti-syndecan-2 antibody is provided; the anti-syndecan-2 antibody comprising a heavy chain variable region and a light chain variable region, wherein the heavy chain variable region comprises an amino acid sequence having at least 90.5% sequence identity to at least one selected from the group consisting of SEQ ID NOs: 1, 3, 5, 7, and 9 and the light chain variable region comprises an amino acid sequence having at least 91.5% sequence identity to at least one selected from the group consisting of SEQ ID NOs: 2, 4, 6, 8, and 10.

In one embodiment, the amino acid sequence comprised within the heavy chain variable region has at least 91%, 92%, 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 1, 3, 5, 7, and 9. In one embodiment, the heavy chain variable region consists of an amino acid sequence having at least 90.5% sequence identity to at least one selected from the group consisting of SEQ ID NOs: 1, 3, 5, 7, and 9. In various embodiments herein, the amino acid sequence of the heavy chain variable region has at least 91%, 92%, 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 1, 3, 5, 7, and 9.

In one embodiment, the amino acid sequence comprised within the light chain variable region has at least 92%, 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 2, 4, 6, 8, and 10. In one embodiment, the light chain variable region consists of an amino acid sequence having at least 91.5% sequence identity to at least one selected from the group consisting of SEQ ID NOs: 2, 4, 6, 8, and 10. In various embodiments therein, the amino acid sequence of the light chain variable region has at least 92%, 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 2, 4, 6, 8, and 10.

In another embodiment, the amino acid sequence of the heavy chain variable region is selected from the group consisting of SEQ ID NOs: 1, 3, 5, 7, and 9 and the amino acid sequence of the light chain variable region is selected from the group consisting of SEQ ID NOs: 2, 4, 6, 8, and 10. In an additional embodiment, the heavy chain variable region consists of an amino acid sequence selected from the group consisting of SEQ ID NOs: 1, 3, 5, 7, and 9 and the light chain variable region consists of an amino acid sequence selected from the group consisting of SEQ ID NOs: 2, 4, 6, 8, and 10. In another embodiment, the amino acid sequence comprised within the heavy chain variable region and the amino acid sequence comprised within the light chain variable region are respectively selected from the group consisting of SEQ ID NOs: 1 and 2, 3 and 4, 5 and 6, 7 and 8, and 9 and 10. In an additional embodiment, the heavy chain variable region and the light chain variable region respectively consist of SEQ ID NOs: 1 and 2, 3 and 4, 5 and 6, 7 and 8, or 9 and 10.

In one embodiment, the anti-syndecan-2 antibody is an IgA, an IgD, an IgE, an IgG, or an IgM.

In another embodiment, the anti-syndecan-2 antibody further comprises a heavy chain constant region and a light chain constant region, wherein the heavy chain constant region comprises an amino acid sequence having at least 90.5% sequence identity to SEQ ID NO: 36 and the light chain constant region comprises an amino acid sequence having at least 91.5% sequence identity to SEQ ID NO: 37. In one embodiment, the heavy chain constant region comprises an amino acid sequence having at least 91%, 92%, 93%, 94%, 95%, 96%, 97%, 98%, 99%, 99.5%, 99.6%, 99.7%, 99.8%, or 99.9% sequence identity, or having 100% sequence identity, to SEQ ID NO: 36. In another embodiment, the light chain constant region comprises an amino acid sequence having at least 92%, 93%, 94%, 95%, 96%, 97%, 98%, 99%, 99.5%, 99.6%, 99.7%, 99.8%, or 99.9% sequence identity, or having 100% sequence identity, to SEQ ID NO: 37. In another embodiment, the heavy chain constant region consists of SEQ ID NO: 36. In an additional embodiment, the light chain constant region consists of SEQ ID NO: 37.

In an embodiment, the heavy chain variable region is fused with an amino acid sequence comprising SEQ ID NO: 38. In another embodiment, the light chain variable region is fused with an amino acid sequence comprising SEQ ID NO: 39. In an embodiment, the N-terminus of the heavy chain variable region is fused to the C-terminus of an amino acid sequence comprising SEQ ID NO: 38. In another embodiment, the N-terminus of the light chain variable region is fused to the C-terminus of an amino acid sequence comprising SEQ ID NO: 39.

In various embodiments, the anti-syndecan-2 antibody is conjugated to a heterologous peptide. In various embodiments, the heterologous peptide may be a cell penetrating peptide, by way of non-limiting example a transactivator of transcription (TAT) peptide, a secretion signal peptide, by way of non-limiting example a preprotrypsin signal sequence or a stability enhancing peptide. The stability enhancing peptide may be any peptide that extends the syndecan-2 disrupting agent's half-life in vivo relative to the syndecan-2 disrupting agent alone.

In various embodiments, the anti-syndecan-2 antibody further comprises a post-translational modification. Non-limiting examples of post-translational modifications include myristoylation, palmitoylation, stearoylation, glycosylation, the addition of heparan sulfate chains, and combinations thereof. One non-limiting example of glycosylation is the addition of heparan sulfate chains to the anti-syndecan-2 antibody.

### Nucleic Acids Encoding Anti-Syndecan-2 Antibodies

In an embodiment, a nucleic acid is provided, the nucleic acid encoding a heavy chain of an anti-syndecan-2 antibody and comprising at least 90.5% sequence identity to at least one polynucleotide of the group consisting of SEQ ID NOs: 40, 42, 44, 46, and 48. In an embodiment, the nucleic acid encoding the heavy chain comprises at least 91%, 92%, 93%, 94%, 95%, 96%, 97%, 98%, 99%, 99.5%, 99.6%, 99.7%, 99.8%, or 99.9% sequence identity, or comprises 100% sequence identity, to at least one polynucleotide of the group consisting of SEQ ID NOs: 40, 42, 44, 46, and 48. In another embodiment, a nucleic acid is provided, the nucleic acid encoding a light chain of an anti-syndecan-2 antibody and comprising at least 91.5% sequence identity to at least one polynucleotide of the group consisting of SEQ ID NOs: 41, 43, 45, 47, and 49. In another embodiment, the nucleic acid encoding the light chain comprises at least 92%, 93%, 94%, 95%, 96%, 97%, 98%, 99%, 99.5%, 99.6%, 99.7%, 99.8%, or 99.9% sequence identity, or comprises 100% sequence identity, to at least one polynucleotide of the group consisting of SEQ ID NOs: 41, 43, 45, 47, and 49. In an embodiment, the nucleic acids encoding the heavy chain and light chain are comprised on a single nucleic acid.

In one embodiment, at least one of the nucleic acids encoding the heavy chain and the light chain of the anti-syndecan-2 antibody is codon-optimized. In an embodiment, the codon-optimized nucleic acid is optimized for a mammal, including a human, a rabbit, a rat, a mouse, a moose, a horse, a donkey, a guinea pig, a hamster, a monkey, a great ape, a chimpanzee, a gorilla, a bonobo, a cow, a cat, a dog, a non-human primate; a bird; a reptile; a fish; an insect, including a fruit fly; a Mollusca, and other forms of vertebrates and invertebrates including Protostomia, Deuterostomia, Chordata, Ambulacraria, Lophotrochazoa, Spiralia, Ecdysozoa, Arthropoda, Tactopoda, Panarthropoda, Gnathifera, Platytrochozoa, Rouphozoa, Gastrotricha, Platyhelminthes, Mesozoa, Annelida, Krytotrochozoa, etc, and cells thereof. In an embodiment, the codon-optimized nucleic acid can be codon optimized for a single-celled organism including a protozoa, a bacterium, and an archea. Codon optimization for humans, veterinary animals (i.e. domesticated animals), and animals used in bench-side and pre-clinical models are preferred. In a preferred embodiment, the codon-optimized nucleic acid sequence is codon-optimized for a cell line or primary cells. In a further preferred embodiment, the codon-optimization is for a CHO cell.

In various embodiments, the nucleic acid encoding the heavy chain and/or the nucleic acid encoding the light chain also encodes an amino acid sequence that affects the intracellular trafficking of the anti-syndecan-2 antibody. Accordingly, in various embodiments, the anti-syndecan-2 antibody comprises an amino acid sequence that affects the intracellular trafficking of the anti-syndecan-2 antibody. In one embodiment, the amino acid sequence that affects the intracellular trafficking of the anti-syndecan-2 antibody signals the cell to package the anti-syndecan-2 antibody into organelles that are trafficked to the cell surface and are released extracellularly. In a preferred embodiment, the nucleotide sequence encoding the amino acid sequence that affects the intracellular trafficking of the anti-syndecan-2 antibody increases the expression of the anti-syndecan-2 antibody by the cells producing the anti-syndecan-2 antibody. Without wishing to be bound by a particular theory, the extracellular release of the anti-syndecan-2 antibody increases the expression of the antibody because the anti-syndecan-2 antibody does not build up in the cell causing toxicity to the cell. In various embodiments, the amino acid sequence used for intracellular trafficking of the anti-syndecan-2 antibody is cleaved. In additional embodiments, the amino acid sequence used for intracellular trafficking of the anti-syndecan-2 antibody is cleaved before, during, or after its release from the cell.

In a preferred embodiment, the nucleic acid encoding the heavy chain and the nucleic acid encoding the light chain are introduced into the same cell, wherein the cell expresses the anti-syndecan-2 antibody which comprises the heavy chain variable region and the light chain variable region.

Methods of Treating Acute Respiratory Distress Syndrome by Administering a Syndecan-2 Disrupting Agent Including an Anti-Syndecan-2 Antibody and/or a Syndecan-2 Disrupting Peptide

As noted above, the present invention also includes methods of using a syndecan-2 disrupting agent. In one aspect, a method of treating acute respiratory distress syndrome (ARDS) in a subject in need thereof is provided; the method comprising administering an effective amount of the syndecan-2 disrupting agent to treat the ARDS. In various embodiments the ARDS is coronavirus disease 2019-induced ARDS (COVID-19-induced ARDS). In various embodiments, the ARDS is caused by the severe acute respiratory syndrome coronavirus 2 (SARS-CoV-2), which is the coronavirus responsible for the COVID-19 disease. In various embodiments, the ARDS is caused by the subject's immune response to the infection of the SARS-CoV-2. In still further various embodiments, the ARDS is caused by the combination of the SARS-CoV-2 and the subject's immune response to the infection of the SARS-CoV-2. In various embodiments, the syndecan-2 disrupting agent comprises, consists essentially of, or consists of: the anti-syndecan-2 antibody, a syndecan-2 disrupting peptide, compositions comprising the anti-syndecan-2 antibody, compositions comprising the syndecan-2 disrupting peptide, or combinations thereof. In various embodiments, the compositions comprising the anti-syndecan-2 antibody and/or the syndecan-2 disrupting peptide further comprises a pharmaceutically acceptable carrier.

In various embodiments, the syndecan-2 disrupting agent comprises a soluble syndecan-2 extracellular domain. In various embodiments, the method comprises administering to the subject an effective amount of a syndecan-2 disrupting peptide comprising an amino acid sequence set forth in SEQ ID NOs: 31, 32, or 33. In various embodiments, the syndecan-2 disrupting agent is a peptide having SEQ ID NO: 31 PAEEDTNVYTEKHSDSLF, corresponding to syndecan-2 extracellular domain amino acids 123-140. In various embodiments, the syndecan-2 disrupting agent is a peptide having SEQ ID NO: 32 PAIKSTDVYTEKHSDNLF corresponding to mouse syndecan-2 region 124-141.

In various embodiments, the syndecan-2 disrupting agent further comprises a heterologous peptide. In various embodiments, the syndecan-2 disrupting peptide further comprises a heterologous peptide. In various embodiments, the heterologous peptide may be a cell penetrating peptide, by way of non-limiting example a transactivator of transcription (TAT) peptide, a secretion signal peptide, by way of non-limiting example a preprotrypsin signal sequence or a stability enhancing peptide. The stability enhancing peptide may be any peptide that extends the syndecan-2 disrupting agent's half-life in vivo relative to the syndecan-2 disrupting agent alone.

In various embodiments, the syndecan-2 disrupting agent further comprises a post-translational modification. Non-limiting examples of post-translational modifications include myristoylation, palmitoylation, stearoylation, glycosylation, and combinations thereof. One non-limiting example of glycosylation is the addition of heparan sulfate chains to the syndecan-2 disrupting agent.

In another aspect, the invention provides method of treating a syndecan-2 associated disease in a subject in need thereof, the method comprising administering to the subject an effective amount of an anti-syndecan-2 antibody to treat the ARDS, the anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

In various embodiments, the syndecan-2 associated disease is acute respiratory distress syndrome (ARDS), stroke, neurological diseases in which the blood brain barrier (BBB) is altered or disrupted (i.e. Parkinson's Diseases, Alzheimer's disease, Huntington's Disease, peripheral neuropathies, traumatic brain injury, epilepsy and multiple sclerosis), neovascular eye diseases (e.g. wet Age-related macular degeneration, Proliferative Diabetic Retinopathy), cardiovascular disease with a component of vascular hyperpermeability (e.g. myocardial infarction, congestive heart failure). In various embodiments, the syndecan-2 associated disease is selected from the group consisting of Blunt trauma injuries, Battlefield injuries, Peripheral vascular disease, Lymphedema and Inflammation-associated edema. In various embodiments, the syndecan-2 associated disease is any condition or situation that requires acceleration of healing when speed is essential (by way of non-limiting example professional sports). In various embodiments, the syndecan-2 associated disease is coronavirus disease 2019-induced ARDS (COVID-19-induced ARDS).

In one embodiment, the subject is a mammal. In a preferred embodiment, the subject is a human.

Appropriate pharmaceutical dosage forms are discussed herein. The regimen of administration may affect what constitutes an effective amount. The therapeutic formulations may be administered to the subject either prior to or after the onset of ARDS. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions comprising the syndecan-2 disrupting agent to a subject, preferably a mammal, more preferably a human, may be carried out using known procedures, at dosages and for periods of time effective to treat ARDS in the subject. An effective amount of the therapeutic compound necessary to achieve a therapeutic effect may vary according to factors such as the state of the disease or disorder in the subject; the age, sex, and weight of the subject; and the ability of the therapeutic compound to treat a disease in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a composition comprising a syndecan-2 disrupting agent is from about 1 and 5,000 mg/kg of body weight/per day. One of ordinary skill in the art would be able to study the relevant factors and make the determination regarding the effective amount of the composition comprising a syndecan-2 disrupting agent without undue experimentation.

Actual dosage levels of the active ingredients in the pharmaceutical composition comprising a syndecan-2 disrupting agent may be varied so as to obtain an amount of the active ingredient (including but not limited to the syndecan-2 disrupting agent) that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions comprising the syndecan-2 disrupting agent are administered to the patient in dosages that range from one to five times per day or more. In other embodiments, the compositions comprising the syndecan-2 disrupting agent are administered to the patient in range of dosages that include, but are not limited to, once every day, every two, days, every three days to once a week, and once every two weeks. It is readily apparent to one skilled in the art that the frequency of administration of the various combination compositions comprising the syndecan-2 disrupting agent varies from individual to individual depending on many factors including, but not limited to, age, disease or disorder to be treated, gender, overall health, and other factors. Thus, the methods disclosed herein should not be construed to be limited to any particular dosage regime and the precise dosage and composition to be administered to any patient is determined by the attending physical taking all other factors about the patient into account.

A syndecan-2 disrupting agent, including an anti-syndecan-2 antibody or an syndecan-2 disrupting peptide, may be administered to the subject in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a syndecan-2 disrupting agent, including an anti-syndecan-2 antibody or an syndecan-2 disrupting peptide, is from about 1 mg and about 2,500 mg. In some embodiments, a dose of a syndecan-2 disrupting agent, including an anti-syndecan-2 antibody or an syndecan-2 disrupting peptide, used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a syndecan-2 disrupting agent, including an anti-syndecan-2 antibody or an syndecan-2 disrupting peptide, alone or in combination with a second pharmaceutical agent; and instructions for using the compound to treat, prevent, or reduce one or more symptoms of a disease in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the syndecan-2 disrupting agent, including the anti-syndecan-2 antibody or the syndecan-2 disrupting peptide, or compositions thereof include intravenous, intra-arterial, subcutaneous, oral, nasal, rectal, intravaginal, parenteral, buccal, sublingual, or topical. The compositions comprising a syndecan-2 disrupting agent may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

Compositions comprising a syndecan-2 disrupting agent also includes a multi-layer tablet comprising a layer providing for the delayed release of one or more syndecan-2 disrupting agents, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

Parenteral Administration

For parenteral administration, compositions comprising the syndecan-2 disrupting agent, including the anti-syndecan-2 antibody or the syndecan-2 disrupting peptide, may be formulated for injection or infusion, for example, intravenous, intra-arterial, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Additional Administration Forms

Additional dosage forms of compositions comprising the syndecan-2 disrupting agent, including the anti-syndecan-2 antibody or the syndecan-2 disrupting peptide, include dosage forms as described in U.S. Pat. Nos. 6,340,475; 6,488,962; 6,451,808; 5,972,389; 5,582,837; and 5,007,790. Additional dosage forms of compositions comprising the syndecan-2 disrupting agent also include dosage forms as described in U.S. Patent Applications Nos. 20030147952; 20030104062; 20030104053; 20030044466; 20030039688; and 20020051820. Additional dosage forms of compositions comprising the syndecan-2 disrupting agent also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of compositions comprising the syndecan-2 disrupting agent, including the anti-syndecan-2 antibody or the syndecan-2 disrupting peptide, may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the methods disclosed herein may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a composition comprising the syndecan-2 disrupting agent depends on the age, sex and weight of the patient, the current medical condition of the patient and the progression of a disease in the patient being treated. The skilled artisan is able to determine appropriate dosages depending on these and other factors.

A suitable dose of a syndecan-2 disrupting agent may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the syndecan-2 disrupting agent is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The composition comprising the syndecan-2 disrupting agent may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### Experimental Examples

The invention is now described with reference to the following Examples. These Examples are provided for the purpose of illustration only, and the invention is not limited to these Examples, but rather encompasses all variations that are evident as a result of the teachings provided herein.

### Materials and Methods

### 1. Project Requirements

Antigen: Mix of three peptides from Syndecan-2 protein synthesized by ProteoGenix:

Cys-KIPAQTKSPEETDK
Cys-DSERKMDPAEEDTN
Cys-PAEEDTNVYTEKHSDSLF

Panning on Premium LiAb-FabMax human naïve library with huge diversity of 5.37×1010 different clones
Screening and validation by ELISA of at least 192 single phage binders against the antigen conjugated to protein carriers (KLH, BSA and OVA)
Aim: to identify binders against: the three peptides separately
Guarantee: at least 3 sequences

### 2. Experimental Contents

### 2.1. Biopanning Rounds

(1) Tubes coated with antigen (mix of the 3 peptides at 1/3 ratio each conjugation: KLH, BSA and OVA)

### (2) Washing

### (3) Blocking

### (4) Washing

(5) Incubation of phage library

### (6) Washing

(7) Elution of phage binders with Glycine-HCl followed by neutralization

### 2.2. Library Depletion

(1) Tubes coated with (5% Milk+1.5% casein sodium) to deplete the library against blocking proteins

### (2) Incubation O/N

### (3) Blocking

### (4) Washing

### (5) Washing

(6) Elution of phage binders and transfer the rest

### 2.3. Determination of the Concentration of Eluted Phages (Output Pfu)

(1) Eluted phages added to E. coli TG1
(2) TG1 poured onto plates and cultured upside down
(3) Calculation of pfu based on number of plaques (ie dead TG1) on plate

### 2.4. Amplification of Eluted Phages

(1) Eluted phages added to TG1
(2) Infection by helper phage and culture
(3) Phage precipitation with PEG/NaCl followed by resuspension
(4) Amplified phages used in next biopanning round and polyclonal ELISA

### 2.5. Polyclonal Phage ELISA

(1) Plate coated with antigen (4.5 μg/mL), No coating plates were used as control

### (2) Washing

### (3) Blocking

### (4) Washing

(5) Incubation of amplified phages from each round

### (6) Washing

(7) Incubation of anti-phage-HRP antibody

### (8) Washing

(9) Incubation of TMB followed by HCl

### (10) Reading at 450 nm

### 2.6. Monoclonal Phage ELISA (Single Phage Binders Screening)

(1) 96 Single TG1 clones picked from plates of step 2.5. and infected with phage helper
(2) Centrifugation and supernatants (ie phages) collected
(3) Plate coated with antigen (Test group: mix of 4 μg/mL; Control group: PBS)

### (4) Washing

### (5) Blocking

### (6) Washing

(7) Incubation of phages from step (2)

### (8) Washing

(9) Incubation of anti-phage-HRP antibody

### (10) Washing

(11) Incubation of TMB followed by HCl

### (12) Reading at 450 nm

### 2.7. Verification Experiment of Superior Clones

(1) Coat the plate with antigen (Test group: 4 μg/mL antigen/PBS; Control group: BSA/PBS, PBS for overnight at 4° C.
(2) Remaining steps are the same as 2.6. (4)˜(12)

Nucleic Acids and Amino Acid Sequences

Table 1 provides exemplary amino acid sequences for anti-syndecan-2 antibodies and nucleic acid sequences encoding the same. Within these sequences, the heavy chain and light chains, including heavy and light chain variable regions and heavy and light chain constant regions, are listed separately. The alpha-numeric name (i.e. 11526A8) identifies those heavy and light chains and heavy and light chain variable regions that were cloned together.

TABLE 1
Amino acid and nucleotide sequences

| SEQ | | |
|---|---|---|
| ID | | |
| NO: | Name | Sequence |
| 1 | 11526A8- | EVQLVQSGGGVVQPGRSLRLSCAASGFTFSRDGMHWVRQAPG |
| | Heavy | KGLEWVAVIWNDGSNKYYADSVKGRFTISRDNSKNTLYLQMN |
| | Chain | SLRAEDTAVYYCARHYGDYGMDVWGQGTTVTVSS |
| | Variable | |
| | Region | |
| 2 | 11526A8- | SSELTQPSSVSMSPGQTARITCSGDVLAKKYARWFQQKPGQAP |
| | Light Chain | VLVIYKDSERPSGIPERFSGSSSGTTVTLTISGVQAEDEADYYCQ |
| | Variable | SADSSGTRWVFGGGTKLTVL |
| | Region | |
| 3 | 11526B9- | QVQLVQSGAEVKKPGASVKVSCKASGYTFTSYYMHWVRQAPG |
| | Heavy | QGLEWMGIINPSGGSTSYAQKFQGRVTMTRDTSTSTVYMELSS |
| | Chain | LRSEDTAVYYCARPRGIRAYGGYSLDYWGQGTLVTVSS |
| | Variable | |
| | Region | |
| 4 | 11526B9- | QTVVTQEPSFSVSPGGTVTLTCGLNSGSVSSRFFPSWYQQTPGQ |
| | Light Chain | PPRTLIYSTRIRSSGVPDRFSGFILGDKAALTITGAQADDESVYYC |
| | Variable | LLYMGSGIWVFGGGTKLTVL |
| | Region | |
| 5 | 11526E1- | QVQLVQSGGGLVQPGGSLRLSCAASGFTFSSYAMSWVRQAPG |
| | Heavy | KGLEWVSAISGSGGSTYYADSVKGRFTISRDNSKNTLYLQMNS |
| | Chain | LRAEDTAVYYCAKHIRGQRYFAYMDVWGKGTLVTVSS |
| | Variable | |
| | Region | |
| 6 | 11526E1- | AIRMTQSPSSVSASVGDRVTITCRASQGISSRLAWYQQKPGKAP |
| | Light Chain | KLLIYAASSLQSGVPSRFSGSGSGTDFTLTISSVQPEDFATYYCQ |
| | Variable | QANSFPLTFGGGTKVDIK |
| | Region | |
| 7 | 11526F3- | QVQLQQSGAEVKKPGASVKVSCKASGYTFTSYYMHWVRQAPG |
| | Heavy | QGLEWMGIINPSGGSTSYAQKFQGRVTMTRDTSTSTVYMELSS |
| | Chain | LRSEDTAVYYCARDRRGGGDYWGQGTLVTVSS |
| | Variable | |
| | Region | |
| 8 | 11526F3- | QAVVTQEPSFSVSPGGTVTLTCGLSSGSVSTTYYPSWYQLTPGQ |
| | Light Chain | APRTLIYNTNTRSSGVPDRFSGSILGNKAALTITGAQADDESYYY |
| | Variable | CLLYMGRGVRVFGGGTKLTVL |
| | Region | |
| 9 | 11526H5- | QVQLVESGGGVVQPGRSLRLSCAASGFTFSSYAVHWVRQAPGK |
| | Heavy | GLEWVAVISYDGSNKYYADSVKGRFTISRDNSKNTLFLQMNSL |
| | Chain | RAEDTAVYYCARRGLRGYAFDIWGQGTLVTVSS |
| | Variable | |
| | Region | |
| 10 | 11526H5- | SYVLTQPSSVSVSPGQTARITCSGDVLAKKYARWFQQKPGQPP |
| | Light Chain | VLLIYKDTERPSGIPERFSGSSSGTTATLTISGAQAEDEADYYCY |
| | Variable | SAADNKGFFGTGTKVTVL |
| | Region | |
| 11 | 11526A8- | EVQLVQSGGGVVQPGRSLRLSCAASGFTFSRDGMHWVRQAPG |
| | Heavy | KGLEWVAVIWNDGSNKYYADSVKGRFTISRDNSKNTLYLQMN |
| | Chain | SLRAEDTAVYYCARHYGDYGMDVWGQGTTVTVSSASTKGPSV |
| | | FPLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPELLGGPSVFLFPPKPKDTLMISRTPE |
| | | VTCVVVDVSHEDPEVKFNWYVDGVEVHNAKTKPREEQYNSTY |
| | | RVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 12 | 11526A8- | SSELTQPSSVSMSPGQTARITCSGDVLAKKYARWFQQKPGQAP |
| | Light Chain | VLVIYKDSERPSGIPERFSGSSSGTTVTLTISGVQAEDEADYYCQ |
| | | SADSSGTRWVFGGGTKLTVLRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 13 | 11526B9- | QVQLVQSGAEVKKPGASVKVSCKASGYTFTSYYMHWVRQAPG |
| | Heavy | QGLEWMGIINPSGGSTSYAQKFQGRVTMTRDTSTSTVYMELSS |
| | Chain | LRSEDTAVYYCARPRGIRAYGGYSLDYWGQGTLVTVSSASTKG |
| | | PSVFPLAPSSKSTSGGTAALGCLVKDYFPEPVTVSWNSGALTSG |
| | | VHTFPAVLQSSGLYSLSSVVTVPSSSLGTQTYICNVNHKPSNTK |
| | | VDKKVEPKSCDKTHTCPPCPAPELLGGPSVFLFPPKPKDTLMISR |
| | | TPEVTCVVVDVSHEDPEVKFNWYVDGVEVHNAKTKPREEQYN |
| | | STYRVVSVLTVLHQDWLNGKEYKCKVSNKALPAPIEKTISKAK |
| | | GQPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 14 | 11526B9- | QTVVTQEPSFSVSPGGTVTLTCGLNSGSVSSRFFPSWYQQTPGQ |
| | Light Chain | PPRTLIYSTRIRSSGVPDRFSGFILGDKAALTITGAQADDESVYYC |
| | | LLYMGSGIWVFGGGTKLTVLRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 15 | 11526E1- | QVQLVQSGGGLVQPGGSLRLSCAASGFTFSSYAMSWVRQAPG |
| | Heavy | KGLEWVSAISGSGGSTYYADSVKGRFTISRDNSKNTLYLQMNS |
| | Chain | LRAEDTAVYYCAKHIRGQRYFAYMDVWGKGTLVTVSSASTKG |
| | | PSVFPLAPSSKSTSGGTAALGCLVKDYFPEPVTVSWNSGALTSG |
| | | VHTFPAVLQSSGLYSLSSVVTVPSSSLGTQTYICNVNHKPSNTK |
| | | VDKKVEPKSCDKTHTCPPCPAPELLGGPSVFLFPPKPKDTLMISR |
| | | TPEVTCVVVDVSHEDPEVKFNWYVDGVEVHNAKTKPREEQYN |
| | | STYRVVSVLTVLHQDWLNGKEYKCKVSNKALPAPIEKTISKAK |
| | | GQPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| 16 | 11526E1- | AIRMTQSPSSVSASVGDRVTITCRASQGISSRLAWYQQKPGKAP |
| | Light Chain | KLLIYAASSLQSGVPSRFSGSGSGTDFTLTISSVQPEDFATYYCQ |
| | | QANSFPLTFGGGTKVDIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| 17 | 11526F3- | QVQLQQSGAEVKKPGASVKVSCKASGYTFTSYYMHWVRQAPG |
| | Heavy | QGLEWMGIINPSGGSTSYAQKFQGRVTMTRDTSTSTVYMELSS |
| | Chain | LRSEDTAVYYCARDRRGGGDYWGQGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPGK |
| 18 | 11526F3- | QAVVTQEPSFSVSPGGTVTLTCGLSSGSVSTTYYPSWYQLTPGQ |
| | Light Chain | APRTLIYNTNTRSSGVPDRFSGSILGNKAALTITGAQADDESYYY |
| | | CLLYMGRGVRVFGGGTKLTVLRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| 19 | 11526H5- | QVQLVESGGGVVQPGRSLRLSCAASGFTFSSYAVHWVRQAPGK |
| | Heavy | GLEWVAVISYDGSNKYYADSVKGRFTISRDNSKNTLFLQMNSL |
| | Chain | RAEDTAVYYCARRGLRGYAFDIWGQGTLVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKKV |
| | | EPKSCDKTHTCPPCPAPELLGGPSVFLFPPKPKDTLMISRTPEVT |
| | | CVVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPSREEMTKNQVSLTCLVKGFYPSDIAVEWESNGQPEN |
| | | NYKTTPPVLDSDGSFFLYSKLTVDKSRWQQGNVFSCSVMHEAL |
| | | HNHYTQKSLSLSPGK |
| 20 | 11526H5- | SYVLTQPSSVSVSPGQTARITCSGDVLAKKYARWFQQKPGQPP |
| | Light Chain | VLLIYKDTERPSGIPERFSGSSSGTTATLTISGAQAEDEADYYCY |
| | | SAADNKGFFGTGTKVTVLRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 21 | 11526A8- | MKHLWFFLLLVAAPRWVLSEVQLVQSGGGVVQPGRSLRLSCA |
| | Heavy | ASGFTFSRDGMHWVRQAPGKGLEWVAVIWNDGSNKYYADSV |
| | Chain- | KGRFTISRDNSKNTLYLQMNSLRAEDTAVYYCARHYGDYGMD |
| | with tag | VWGQGTTVTVSSASTKGPSVFPLAPSSKSTSGGTAALGCLVKD |
| | (underlined) | YFPEPVTVSWNSGALTSGVHTFPAVLQSSGLYSLSSVVTVPSSSL |
| | | GTQTYICNVNHKPSNTKVDKKVEPKSCDKTHTCPPCPAPELLGG |
| | | PSVFLFPPKPKDTLMISRTPEVTCVVVDVSHEDPEVKFNWYVDG |
| | | VEVHNAKTKPREEQYNSTYRVVSVLTVLHQDWLNGKEYKCKV |
| | | SNKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 22 | 11526A8- | MVLQTQVFISLLLWISGAYGSSELTQPSSVSMSPGQTARITCSGD |
| | Light | VLAKKYARWFQQKPGQAPVLVIYKDSERPSGIPERFSGSSSGTT |
| | Chain- | VTLTISGVQAEDEADYYCQSADSSGTRWVFGGGTKLTVLRTVA |
| | with tag | APSVFIFPPSDEQLKSGTASVVCLLNNFYPREAKVQWKVDNAL |
| | (underlined) | QSGNSQESVTEQDSKDSTYSLSSTLTLSKADYEKHKVYACEVT |
| | | HQGLSSPVTKSFNRGEC |
| 23 | 11526B9- | MKHLWFFLLLVAAPRWVLSQVQLVQSGAEVKKPGASVKVSCK |
| | Heavy | ASGYTFTSYYMHWVRQAPGQGLEWMGIINPSGGSTSYAQKFQ |
| | Chain- | GRVTMTRDTSTSTVYMELSSLRSEDTAVYYCARPRGIRAYGGY |
| | with tag | SLDYWGQGTLVTVSSASTKGPSVFPLAPSSKSTSGGTAALGCLV |
| | (underlined) | KDYFPEPVTVSWNSGALTSGVHTFPAVLQSSGLYSLSSVVTVPS |
| | | SSLGTQTYICNVNHKPSNTKVDKKVEPKSCDKTHTCPPCPAPEL |
| | | LGGPSVFLFPPKPKDTLMISRTPEVTCVVVDVSHEDPEVKFNWY |
| | | VDGVEVHNAKTKPREEQYNSTYRVVSVLTVLHQDWLNGKEYK |
| | | CKVSNKALPAPIEKTISKAKGQPREPQVYTLPPSREEMTKNQVS |
| | | LTCLVKGFYPSDIAVEWESNGQPENNYKTTPPVLDSDGSFFLYS |
| | | KLTVDKSRWQQGNVFSCSVMHEALHNHYTQKSLSLSPGK |
| 24 | 11526B9- | MVLQTQVFISLLLWISGAYGQTVVTQEPSFSVSPGGTVTLTCGL |
| | Light | NSGSVSSRFFPSWYQQTPGQPPRTLIYSTRIRSSGVPDRFSGFILG |
| | Chain- | DKAALTITGAQADDESVYYCLLYMGSGIWVFGGGTKLTVLRTV |
| | with tag | AAPSVFIFPPSDEQLKSGTASVVCLLNNFYPREAKVQWKVDNA |
| | (underlined) | LQSGNSQESVTEQDSKDSTYSLSSTLTLSKADYEKHKVYACEVT |
| | | HQGLSSPVTKSFNRGEC |
| 25 | 11526E1- | MKHLWFFLLLVAAPRWVLSQVQLVQSGGGLVQPGGSLRLSCA |
| | Heavy | ASGFTFSSYAMSWVRQAPGKGLEWVSAISGSGGSTYYADSVKG |
| | Chain- | RFTISRDNSKNTLYLQMNSLRAEDTAVYYCAKHIRGQRYFAYM |
| | with tag | DVWGKGTLVTVSSASTKGPSVFPLAPSSKSTSGGTAALGCLVK |
| | (underlined) | DYFPEPVTVSWNSGALTSGVHTFPAVLQSSGLYSLSSVVTVPSS |
| | | SLGTQTYICNVNHKPSNTKVDKKVEPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKC |
| | | KVSNKALPAPIEKTISKAKGQPREPQVYTLPPSREEMTKNQVSL |
| | | TCLVKGFYPSDIAVEWESNGQPENNYKTTPPVLDSDGSFFLYSK |
| | | LTVDKSRWQQGNVFSCSVMHEALHNHYTQKSLSLSPGK |
| 26 | 11526E1- | MVLQTQVFISLLLWISGAYGAIRMTQSPSSVSASVGDRVTITCR |
| | Chain- | ASQGISSRLAWYQQKPGKAPKLLIYAASSLQSGVPSRFSGSGSG |
| | with tag | TDFTLTISSVQPEDFATYYCQQANSFPLTFGGGTKVDIKRTVAAP |
| | (underlined) | SVFIFPPSDEQLKSGTASVVCLLNNFYPREAKVQWKVDNALQS |
| | Light | GNSQESVTEQDSKDSTYSLSSTLTLSKADYEKHKVYACEVTHQ |
| | | GLSSPVTKSFNRGEC |
| 27 | 11526F3- | MKHLWFFLLLVAAPRWVLSQVQLQQSGAEVKKPGASVKVSCK |
| | Chain- | ASGYTFTSYYMHWVRQAPGQGLEWMGIINPSGGSTSYAQKFQ |
| | with tag | GRVTMTRDTSTSTVYMELSSLRSEDTAVYYCARDRRGGGDYW |
| | (underlined) | GQGTLVTVSSASTKGPSVFPLAPSSKSTSGGTAALGCLVKDYFP |
| | Heavy | EPVTVSWNSGALTSGVHTFPAVLQSSGLYSLSSVVTVPSSSLGT |
| | | QTYICNVNHKPSNTKVDKKVEPKSCDKTHTCPPCPAPELLGGPS |
| | | VFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 28 | 11526F3- | MVLQTQVFISLLLWISGAYGQAVVTQEPSFSVSPGGTVTLTCGL |
| | Chain- | SSGSVSTTYYPSWYQLTPGQAPRTLIYNTNTRSSGVPDRFSGSIL |
| | with tag | GNKAALTITGAQADDESYYYCLLYMGRGVRVFGGGTKLTVLR |
| | (underlined) | TVAAPSVFIFPPSDEQLKSGTASVVCLLNNFYPREAKVQWKVD |
| | Light | NALQSGNSQESVTEQDSKDSTYSLSSTLTLSKADYEKHKVYAC |
| | | EVTHQGLSSPVTKSFNRGEC |
| 29 | 11526H5- | MKHLWFFLLLVAAPRWVLSQVQLVESGGGVVQPGRSLRLSCA |
| | Chain- | ASGFTFSSYAVHWVRQAPGKGLEWVAVISYDGSNKYYADSVK |
| | with tag | GRFTISRDNSKNTLFLQMNSLRAEDTAVYYCARRGLRGYAFDI |
| | (underlined) | WGQGTLVTVSSASTKGPSVFPLAPSSKSTSGGTAALGCLVKDYF |
| | Heavy | PEPVTVSWNSGALTSGVHTFPAVLQSSGLYSLSSVVTVPSSSLGT |
| | | QTYICNVNHKPSNTKVDKKVEPKSCDKTHTCPPCPAPELLGGPS |
| | | VFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSREEMTKNQVSLTCL |
| | | VKGFYPSDIAVEWESNGQPENNYKTTPPVLDSDGSFFLYSKLTV |
| | | DKSRWQQGNVFSCSVMHEALHNHYTQKSLSLSPGK |
| 30 | 11526H5- | MVLQTQVFISLLLWISGAYGSYVLTQPSSVSVSPGQTARITCSGD |
| | Chain- | VLAKKYARWFQQKPGQPPVLLIYKDTERPSGIPERFSGSSSGTT |
| | with tag | ATLTISGAQAEDEADYYCYSAADNKGFFGTGTKVTVLRTVAAP |
| | (underlined) | SVFIFPPSDEQLKSGTASVVCLLNNFYPREAKVQWKVDNALQS |
| | Light | GNSQESVTEQDSKDSTYSLSSTLTLSKADYEKHKVYACEVTHQ |
| | | GLSSPVTKSFNRGEC |
| 31 | Syndecan-2 | PAEEDTNVYTEKHSDSLF |
| | disrupting | |
| | peptide | |
| | human | |
| 32 | Syndecan-2 | PAIKSTDVYTEKHSDNLF |
| | disrupting | |
| | peptide | |
| | mouse | |
| 33 | Syndecan-2 | MYLDNSSIEEASGVYPIDDDDYASASGSGADEDVESPELTTSRP |
| | disrupting | LPKILLTSAAPKVETTTLNIQNKIPAQTKSPEETDKEKVHLSDSE |
| | peptide | RKMDPAEEDTNVYTEKHSDSLFKRTEVLAAVIAGGVIGFLFAIF |
| | human | LILL |
| 34 | Human | MRRAWILLTLGLVACVSAESRAELTSDKDMYLDNSSIEEASGV |
| | syndecan-2 | YPIDDDDYASASGSGADEDVESPELTTSRPLPKILLTSAAPKVET |
| | | TTLNIQNKIPAQTKSPEETDKEKVHLSDSERKMDPAEEDTNVYT |
| | | EKHSDSLFKRTEVLAAVIAGGVIGFLFAIFLILLLVYRMRKKDEG |
| | | SYDLGERKPSSAAYQKAPTKEFYA |
| 35 | Mouse | MQRAWILLTLGLMACVSAETRTELTSDKDMYLDNSSIEEASGV |
| | syndecan-2 | YPIDDDDYSSASGSGADEDIESPVLTTSQLIPRIPLTSAASPKVET |
| | | MTLKTQSITPAQTESPEETDKEEVDISEAEEKLGPAIKSTDVYTE |
| | | KHSDNLFKRTEVLAAVIAGGVIGFLFAIFLILLLVYRMRKKDEGS |
| | | YDLGERKPSSAAYQKAPTKEFYA |
| 36 | Anti- | ASTKGPSVFPLAPSSKSTSGGTAALGCLVKDYFPEPVTVSWNSG |
| | Syndecan 2 | ALTSGVHTFPAVLQSSGLYSLSSVVTVPSSSLGTQTYICNVNHKP |
| | IgG heavy | SNTKVDKKVEPKSCDKTHTCPPCPAPELLGGPSVFLFPPKPKDT |
| | chain | LMISRTPEVTCVVVDVSHEDPEVKFNWYVDGVEVHNAKTKPR |
| | constant | EEQYNSTYRVVSVLTVLHQDWLNGKEYKCKVSNKALPAPIEKT |
| | region | ISKAKGQPREPQVYTLPPSREEMTKNQVSLTCLVKGFYPSDIAV |
| | | EWESNGQPENNYKTTPPVLDSDGSFFLYSKLTVDKSRWQQGNV |
| | | FSCSVMHEALHNHYTQKSLSLSPGK |
| 37 | Anti- | RTVAAPSVFIFPPSDEQLKSGTASVVCLLNNFYPREAKVQWKVD |
| | Syndecan 2 | NALQSGNSQESVTEQDSKDSTYSLSSTLTLSKADYEKHKVYAC |
| | IgG light | EVTHQGLSSPVTKSFNRGEC |
| | chain | |
| | constant | |
| | region | |
| 38 | Anti- | MKHLWFFLLLVAAPRWVL |
| | Syndecan 2 | |
| | heavy chain | |
| | tag | |
| 39 | Anti- | MVLQTQVFISLLLWISGAY |
| | Syndecan 2 | |
| | light chain | |
| | tag | |
| 40 | 11256A8- | GAATTCGCCGCCACCATGAAGCACCTGTGGTTCTTCCTGCTG |
| | HC cDNA | CTGGTGGCCGCCCCCCGGTGGGTGCTGTCCGAGGTGCAGCTG |
| | | GTGCAGTCCGGCGGCGGAGTGGTGCAGCCTGGAAGGAGCCT |
| | | GAGGCTGTCCTGCGCTGCTTCTGGCTTCACCTTCAGCCGGGA |
| | | CGGCATGCACTGGGTGAGGCAGGCCCCCGGAAAGGGCCTGG |
| | | AGTGGGTGGCTGTGATCTGGAATGACGGCTCCAACAAGTACT |
| | | ACGCCGATTCCGTGAAGGGCCGGTTCACCATCAGCCGGGAC |
| | | AATTCCAAGAATACCCTGTACCTGCAGATGAATAGCCTGCGG |
| | | GCCGAGGATACCGCTGTGTATTACTGTGCTCGGCACTATGGC |
| | | GACTACGGCATGGACGTGTGGGGCCAGGGCACCACCGTGAC |
| | | CGTGAGCTCCGCTAGCACCAAGGGACCTTCTGTGTTCCCTCT |
| | | GGCTCCTTCTTCTAAGTCCACTTCCGGTGGTACAGCAGCTCTG |
| | | GGTTGTCTGGTGAAGGATTACTTCCCAGAACCAGTGACTGTG |
| | | TCCTGGAACTCCGGAGCTCTGACTTCTGGAGTGCATACTTTC |
| | | CCAGCAGTGCTGCAATCTAGCGGACTGTACTCTCTGTCTTCC |
| | | GTGGTGACTGTGCCTTCTTCTTCCCTGGGGACTCAAACTTACA |
| | | TCTGCAACGTGAACCACAAGCCCTCCAACACCAAGGTGGAC |
| | | AAGAAGGTGGAGCCAAAGAGCTGCGATAAGACCCACACCTG |
| | | TCCACCTTGTCCAGCTCCAGAACTGCTGGGTGGGCCTTCTGT |
| | | GTTTCTGTTCCCACCTAAGCCAAAGGATACCCTGATGATCTC |
| | | TAGGACCCCAGAAGTGACCTGTGTGGTCGTCGATGTGTCTCA |
| | | TGAAGACCCTGAAGTGAAGTTCAACTGGTACGTGGACGGGG |
| | | TGGAAGTGCATAACGCAAAGACCAAGCCCAGGGAAGAGCAA |
| | | TACAACTCCACCTACAGGGTGGTCTCCGTCCTGACAGTCCTG |
| | | CATCAGGATTGGCTGAACGGCAAGGAGTACAAGTGCAAGGT |
| | | CTCCAATAAAGCCCTGCCTGCCCCTATCGAGAAAACCATTAG |
| | | CAAAGCCAAAGGCCAGCCCAGGGAGCCCCAGGTCTATACAC |
| | | TGCCCCCCAGCAGGGAGGAGATGACAAAAAATCAGGTCAGC |
| | | CTGACATGCCTGGTCAAAGGCTTTTATCCCAGCGACATTGCC |
| | | GTCGAGTGGGAGTCCAATGGCCAGCCCGAGAATAATTATAA |
| | | AACAACACCCCCCGTCCTGGACAGCGACGGCAGCTTTTTTCT |
| | | GTATAGCAAACTGACAGTCGATAAAAGCAGGTGGCAGCAGG |
| | | GCAATGTCTTTTCCTGCAGCGTCATGCACGAGGCCCTGCACA |
| | | ATCACTATACTCAGAAAAGCCTGAGCCTGTCCCCCGGGAAAT |
| | | GAGCGGCCGC |
| 41 | 11256A8- | GAATTCGCCGCCACCATGGTGCTGCAGACCCAGGTGTTCATC |
| | LC cDNA | TCCCTGCTGCTGTGGATCAGCGGCGCCTACGGCTCCTCCGAG |
| | | CTGACCCAGCCTAGCTCCGTGAGCATGTCCCCTGGCCAGACC |
| | | GCCAGGATCACCTGCTCCGGCGATGTGCTGGCTAAGAAGTAC |
| | | GCTAGGTGGTTCCAGCAGAAGCCCGGCCAGGCTCCCGTGCTG |
| | | GTGATCTACAAGGATAGCGAGCGGCCCTCCGGCATCCCTGAG |
| | | AGGTTCAGCGGCAGCAGCAGCGGCACCACCGTGACCCTGAC |
| | | CATCTCCGGCGTGCAGGCCGAGGACGAGGCTGACTACTATTG |
| | | CCAGTCCGCTGATAGCTCCGGCACCAGGTGGGTGTTCGGCGG |
| | | CGGAACCAAGCTGACCGTGCTGCGTACGGTGGCTGCACCTTC |
| | | TGTGTTCATCTTCCCTCCATCTGATGAGCAGCTGAAGTCTGG |
| | | AACCGCATCTGTCGTCTGTCTGCTGAACAACTTTTACCCCAG |
| | | GGAGGCTAAGGTCCAATGGAAGGTGGACAACGCCCTGCAGT |
| | | CTGGTAATAGCCAGGAAAGCGTGACCGAACAGGATTCCAAG |
| | | GACTCCACCTACTCCCTGTCCTCCACACTGACACTGAGCAAA |
| | | GCCGACTATGAAAAGCACAAAGTGTATGCCTGCGAGGTCAC |
| | | TCATCAGGGCCTGTCCAGCCCCGTGACTAAAAGCTTTAATAG |
| | | GGGGGAGTGCTGAGCGGCCGC |
| 42 | 11256B9- | GAATTCGCCGCCACCATGAAGCACCTGTGGTTCTTCCTGCTG |
| | HC cDNA | CTGGTGGCTGCTCCCCGGTGGGTGCTGAGCCAGGTGCAGCTG |
| | | GTGCAGAGCGGCGCTGAGGTGAAGAAGCCTGGCGCTTCTGT |
| | | GAAGGTGTCCTGTAAGGCCAGCGGCTATACCTTCACCTCCTA |
| | | CTACATGCACTGGGTGAGGCAGGCTCCTGGCCAGGGCCTGG |
| | | AGTGGATGGGCATCATCAATCCCAGCGGCGGCTCCACCTCCT |
| | | ACGCTCAGAAGTTCCAGGGCCGGGTGACCATGACCAGGGAC |
| | | ACCTCCACCAGCACCGTGTACATGGAGCTGTCCTCCCTGCGG |
| | | AGCGAGGATACCGCCGTGTACTATTGTGCCCGGCCTAGGGGC |
| | | ATCAGGGCCTACGGAGGCTACAGCCTGGATTATTGGGGCCA |
| | | GGGCACCCTGGTGACCGTGTCCTCCGCTAGCACCAAGGGACC |
| | | TTCTGTGTTCCCTCTGGCTCCTTCTTCTAAGTCCACTTCCGGT |
| | | GGTACAGCAGCTCTGGGTTGTCTGGTGAAGGATTACTTCCCA |
| | | GAACCAGTGACTGTGTCCTGGAACTCCGGAGCTCTGACTTCT |
| | | GGAGTGCATACTTTCCCAGCAGTGCTGCAATCTAGCGGACTG |
| | | TACTCTCTGTCTTCCGTGGTGACTGTGCCTTCTTCTTCCCTGG |
| | | GGACTCAAACTTACATCTGCAACGTGAACCACAAGCCCTCCA |
| | | ACACCAAGGTGGACAAGAAGGTGGAGCCAAAGAGCTGCGAT |
| | | AAGACCCACACCTGTCCACCTTGTCCAGCTCCAGAACTGCTG |
| | | GGTGGGCCTTCTGTGTTTCTGTTCCCACCTAAGCCAAAGGAT |
| | | ACCCTGATGATCTCTAGGACCCCAGAAGTGACCTGTGTGGTC |
| | | GTCGATGTGTCTCATGAAGACCCTGAAGTGAAGTTCAACTGG |
| | | TACGTGGACGGGGTGGAAGTGCATAACGCAAAGACCAAGCC |
| | | CAGGGAAGAGCAATACAACTCCACCTACAGGGTGGTCTCCG |
| | | TCCTGACAGTCCTGCATCAGGATTGGCTGAACGGCAAGGAGT |
| | | ACAAGTGCAAGGTCTCCAATAAAGCCCTGCCTGCCCCTATCG |
| | | AGAAAACCATTAGCAAAGCCAAAGGCCAGCCCAGGGAGCCC |
| | | CAGGTCTATACACTGCCCCCCAGCAGGGAGGAGATGACAAA |
| | | AAATCAGGTCAGCCTGACATGCCTGGTCAAAGGCTTTTATCC |
| | | CAGCGACATTGCCGTCGAGTGGGAGTCCAATGGCCAGCCCG |
| | | AGAATAATTATAAAACAACACCCCCCGTCCTGGACAGCGAC |
| | | GGCAGCTTTTTTCTGTATAGCAAACTGACAGTCGATAAAAGC |
| | | AGGTGGCAGCAGGGCAATGTCTTTTCCTGCAGCGTCATGCAC |
| | | GAGGCCCTGCACAATCACTATACTCAGAAAAGCCTGAGCCTG |
| | | TCCCCCGGGAAATGAGCGGCCGC |
| 43 | 11256B9-LC | GAATTCGCCGCCACCATGGTGCTGCAGACCCAGGTGTTCATC |
| | cDNA | TCCCTGCTGCTGTGGATCTCCGGCGCCTATGGCCAGACCGTG |
| | | GTGACCCAGGAGCCTAGCTTCTCCGTGTCCCCTGGCGGCACC |
| | | GTGACCCTGACCTGCGGACTGAACAGCGGCAGCGTGTCCAG |
| | | CAGGTTCTTCCCTAGCTGGTACCAGCAGACCCCCGGCCAGCC |
| | | CCCAAGAACCCTGATCTATAGCACCAGGATCCGGAGCAGCG |
| | | GCGTGCCTGACAGGTTCAGCGGCTTCATCCTGGGCGACAAGG |
| | | CCGCTCTGACCATCACCGGCGCCCAGGCTGACGATGAGAGC |
| | | GTGTATTACTGTCTGCTGTACATGGGCTCCGGCATCTGGGTG |
| | | TTCGGCGGCGGAACCAAGCTGACCGTGCTGCGTACGGTGGCT |
| | | GCACCTTCTGTGTTCATCTTCCCTCCATCTGATGAGCAGCTGA |
| | | AGTCTGGAACCGCATCTGTCGTCTGTCTGCTGAACAACTTTT |
| | | ACCCCAGGGAGGCTAAGGTCCAATGGAAGGTGGACAACGCC |
| | | CTGCAGTCTGGTAATAGCCAGGAAAGCGTGACCGAACAGGA |
| | | TTCCAAGGACTCCACCTACTCCCTGTCCTCCACACTGACACT |
| | | GAGCAAAGCCGACTATGAAAAGCACAAAGTGTATGCCTGCG |
| | | AGGTCACTCATCAGGGCCTGTCCAGCCCCGTGACTAAAAGCT |
| | | TTAATAGGGGGGAGTGCTGAGCGGCCGC |

| | | |
|---|---|---|
| 44 | 11256E1- | GAATTCGCCGCCACCATGAAGCACCTGTGGTTCTTCCTGCTG |
| | HCcDNA | CTGGTGGCTGCCCCCCGGTGGGTTCTGTCCCAGGTGCAGCTG |
| | | GTGCAGTCCGGCGGCGGACTGGTGCAGCCTGGAGGATCCCT |
| | | GAGGCTGAGCTGCGCTGCCAGCGGCTTCACCTTCAGCTCCTA |
| | | CGCTATGTCCTGGGTGCGGCAGGCCCCCGGAAAGGGACTGG |
| | | AGTGGGTGAGCGCCATCTCCGGCAGCGGCGGAAGCACCTAC |
| | | TACGCCGACTCCGTGAAGGGCAGGTTCACCATCTCCAGGGAT |
| | | AACTCCAAGAACACCCTGTATCTGCAGATGAACAGCCTGCGG |
| | | GCTGAGGATACCGCCGTGTATTACTGCGCCAAGCACATCCGG |
| | | GGCCAGAGGTACTTCGCCTACATGGATGTGTGGGGCAAGGG |
| | | CACCCTGGTGACCGTGTCCTCCGCTAGCACCAAGGGACCTTC |
| | | TGTGTTCCCTCTGGCTCCTTCTTCTAAGTCCACTTCCGGTGGT |
| | | ACAGCAGCTCTGGGTTGTCTGGTGAAGGATTACTTCCCAGAA |
| | | CCAGTGACTGTGTCCTGGAACTCCGGAGCTCTGACTTCTGGA |
| | | GTGCATACTTTCCCAGCAGTGCTGCAATCTAGCGGACTGTAC |
| | | TCTCTGTCTTCCGTGGTGACTGTGCCTTCTTCTTCCCTGGGGA |
| | | CTCAAACTTACATCTGCAACGTGAACCACAAGCCCTCCAACA |
| | | CCAAGGTGGACAAGAAGGTGGAGCCAAAGAGCTGCGATAAG |
| | | ACCCACACCTGTCCACCTTGTCCAGCTCCAGAACTGCTGGGT |
| | | GGGCCTTCTGTGTTTCTGTTCCCACCTAAGCCAAAGGATACC |
| | | CTGATGATCTCTAGGACCCCAGAAGTGACCTGTGTGGTCGTC |
| | | GATGTGTCTCATGAAGACCCTGAAGTGAAGTTCAACTGGTAC |
| | | GTGGACGGGGTGGAAGTGCATAACGCAAAGACCAAGCCCAG |
| | | GGAAGAGCAATACAACTCCACCTACAGGGTGGTCTCCGTCCT |
| | | GACAGTCCTGCATCAGGATTGGCTGAACGGCAAGGAGTACA |
| | | AGTGCAAGGTCTCCAATAAAGCCCTGCCTGCCCCTATCGAGA |
| | | AAACCATTAGCAAAGCCAAAGGCCAGCCCAGGGAGCCCCAG |
| | | GTCTATACACTGCCCCCCAGCAGGGAGGAGATGACAAAAAA |
| | | TCAGGTCAGCCTGACATGCCTGGTCAAAGGCTTTTATCCCAG |
| | | CGACATTGCCGTCGAGTGGGAGTCCAATGGCCAGCCCGAGA |
| | | ATAATTATAAAACAACACCCCCCGTCCTGGACAGCGACGGC |
| | | AGCTTTTTTCTGTATAGCAAACTGACAGTCGATAAAAGCAGG |
| | | TGGCAGCAGGGCAATGTCTTTTCCTGCAGCGTCATGCACGAG |
| | | GCCCTGCACAATCACTATACTCAGAAAAGCCTGAGCCTGTCC |
| | | CCCGGGAAATGAGCGGCCGC |
| 45 | 11256E1-LC | GAATTCGCCGCCACCATGGTGCTGCAGACCCAGGTGTTCATC |
| | cDNA | AGCCTGCTGCTGTGGATCTCCGGCGCTTATGGCGCTATCCGG |
| | | ATGACCCAGAGCCCCAGCTCCGTGTCCGCTAGCGTGGGCGAC |
| | | AGGGTGACCATCACCTGTAGGGCCTCCCAGGGCATCTCCTCC |
| | | CGGCTGGCTTGGTATCAGCAGAAGCCCGGCAAGGCTCCCAA |
| | | GCTGCTGATCTATGCTGCCAGCTCCCTGCAGAGCGGCGTGCC |
| | | TTCCCGGTTCAGCGGCTCCGGATCCGGCACCGACTTCACCCT |
| | | GACCATCTCCAGCGTGCAGCCTGAGGACTTCGCTACCTACTA |
| | | TTGTCAGCAGGCCAATTCCTTCCCTCTGACCTTCGGCGGCGG |
| | | CACCAAGGTGGACATCAAGCGTACGGTGGCTGCACCTTCTGT |
| | | GTTCATCTTCCCTCCATCTGATGAGCAGCTGAAGTCTGGAAC |
| | | CGCATCTGTCGTCTGTCTGCTGAACAACTTTTACCCCAGGGA |
| | | GGCTAAGGTCCAATGGAAGGTGGACAACGCCCTGCAGTCTG |
| | | GTAATAGCCAGGAAAGCGTGACCGAACAGGATTCCAAGGAC |
| | | TCCACCTACTCCCTGTCCTCCACACTGACACTGAGCAAAGCC |
| | | GACTATGAAAAGCACAAAGTGTATGCCTGCGAGGTCACTCAT |
| | | CAGGGCCTGTCCAGCCCCGTGACTAAAAGCTTTAATAGGGGG |
| | | GAGTGCTGAGCGGCCGC |
| 46 | 11256F3- | GAATTCGCCGCCACCATGAAGCACCTGTGGTTCTTCCTGCTG |
| | HC cDNA | CTGGTGGCTGCCCCTAGGTGGGTGCTGAGCCAGGTGCAGCTG |
| | | CAGCAGAGCGGCGCTGAGGTGAAGAAGCCCGGCGCCAGCGT |
| | | GAAGGTGAGCTGTAAGGCTTCCGGCTACACCTTCACCTCCTA |
| | | CTACATGCACTGGGTGAGGCAGGCTCCCGGCCAGGGACTGG |
| | | AGTGGATGGGCATCATCAACCCTAGCGGCGGCTCCACCTCCT |
| | | ACGCCCAGAAGTTCCAGGGCAGGGTGACCATGACCAGGGAC |
| | | ACCTCCACCAGCACCGTGTACATGGAGCTGTCCAGCCTGAGG |
| | | AGCGAGGATACCGCTGTGTATTACTGTGCTCGGGACCGGCGG |
| | | GGCGGCGGAGACTACTGGGGACAGGGAACCCTGGTGACCGT |
| | | GAGCTCCGCTAGCACCAAGGGACCTTCTGTGTTCCCTCTGGC |
| | | TCCTTCTTCTAAGTCCACTTCCGGTGGTACAGCAGCTCTGGGT |
| | | TGTCTGGTGAAGGATTACTTCCCAGAACCAGTGACTGTGTCC |
| | | TGGAACTCCGGAGCTCTGACTTCTGGAGTGCATACTTTCCCA |
| | | GCAGTGCTGCAATCTAGCGGACTGTACTCTCTGTCTTCCGTG |
| | | GTGACTGTGCCTTCTTCTTCCCTGGGGACTCAAACTTACATCT |
| | | GCAACGTGAACCACAAGCCCTCCAACACCAAGGTGGACAAG |
| | | AAGGTGGAGCCAAAGAGCTGCGATAAGACCCACACCTGTCC |
| | | ACCTTGTCCAGCTCCAGAACTGCTGGGTGGGCCTTCTGTGTTT |
| | | CTGTTCCCACCTAAGCCAAAGGATACCCTGATGATCTCTAGG |
| | | ACCCCAGAAGTGACCTGTGTGGTCGTCGATGTGTCTCATGAA |
| | | GACCCTGAAGTGAAGTTCAACTGGTACGTGGACGGGGTGGA |
| | | AGTGCATAACGCAAAGACCAAGCCCAGGGAAGAGCAATACA |
| | | ACTCCACCTACAGGGTGGTCTCCGTCCTGACAGTCCTGCATC |
| | | AGGATTGGCTGAACGGCAAGGAGTACAAGTGCAAGGTCTCC |
| | | AATAAAGCCCTGCCTGCCCCTATCGAGAAAACCATTAGCAAA |
| | | GCCAAAGGCCAGCCCAGGGAGCCCCAGGTCTATACACTGCC |
| | | CCCCAGCAGGGAGGAGATGACAAAAAATCAGGTCAGCCTGA |
| | | CATGCCTGGTCAAAGGCTTTTATCCCAGCGACATTGCCGTCG |
| | | AGTGGGAGTCCAATGGCCAGCCCGAGAATAATTATAAAACA |
| | | ACACCCCCCGTCCTGGACAGCGACGGCAGCTTTTTTCTGTAT |
| | | AGCAAACTGACAGTCGATAAAAGCAGGTGGCAGCAGGGCAA |
| | | TGTCTTTTCCTGCAGCGTCATGCACGAGGCCCTGCACAATCA |
| | | CTATACTCAGAAAAGCCTGAGCCTGTCCCCCGGGAAATGAGC |
| | | GGCCGC |
| 47 | 11256F3-LC | GAATTCGCCGCCACCATGGTGCTGCAGACCCAGGTGTTCATC |
| | cDNA | TCCCTGCTGCTGTGGATCTCCGGCGCTTATGGCCAGGCTGTG |
| | | GTGACCCAGGAGCCTTCCTTCAGCGTGTCCCCTGGCGGCACC |
| | | GTGACCCTGACCTGTGGCCTGTCCAGCGGCAGCGTGAGCACC |
| | | ACCTACTACCCTTCCTGGTACCAGCTGACCCCTGGCCAGGCC |
| | | CCTCGGACACTGATCTATAATACCAACACCCGGAGCAGCGGC |
| | | GTGCCCGATCGGTTCAGCGGCTCCATCCTGGGCAATAAGGCT |
| | | GCTCTGACCATCACCGGCGCTCAGGCTGACGATGAGAGCTAT |
| | | TACTACTGTCTGCTGTACATGGGCAGGGGCGTGCGGGTGTTC |
| | | GGCGGAGGAACCAAGCTGACCGTGCTGCGTACGGTGGCTGC |
| | | ACCTTCTGTGTTCATCTTCCCTCCATCTGATGAGCAGCTGAAG |
| | | TCTGGAACCGCATCTGTCGTCTGTCTGCTGAACAACTTTTACC |
| | | CCAGGGAGGCTAAGGTCCAATGGAAGGTGGACAACGCCCTG |
| | | CAGTCTGGTAATAGCCAGGAAAGCGTGACCGAACAGGATTC |
| | | CAAGGACTCCACCTACTCCCTGTCCTCCACACTGACACTGAG |
| | | CAAAGCCGACTATGAAAAGCACAAAGTGTATGCCTGCGAGG |
| | | TCACTCATCAGGGCCTGTCCAGCCCCGTGACTAAAAGCTTTA |
| | | ATAGGGGGGAGTGCTGAGCGGCCGC |
| 48 | 11256H5- | GAATTCGCCGCCACCATGAAGCACCTGTGGTTCTTCCTGCTG |
| | HC cDNA | CTGGTGGCTGCCCCTCGGTGGGTGCTGTCCCAGGTGCAGCTG |
| | | GTGGAGAGCGGCGGCGGAGTGGTGCAGCCAGGAAGAAGCCT |
| | | GAGGCTGAGCTGTGCCGCTTCCGGCTTCACCTTCAGCTCCTA |
| | | CGCTGTGCACTGGGTGCGGCAGGCTCCCGGAAAGGGCCTGG |
| | | AGTGGGTGGCTGTGATCTCCTACGATGGCTCCAACAAGTACT |
| | | ATGCCGACTCCGTGAAGGGCCGGTTCACCATCTCCCGGGATA |
| | | ATTCCAAGAACACCCTGTTCCTGCAGATGAATAGCCTGAGGG |
| | | CCGAGGACACCGCTGTGTACTATTGCGCCCGGAGGGGCCTGC |
| | | GGGGCTATGCCTTCGATATCTGGGGCCAGGGCACCCTGGTGA |
| | | CCGTGTCCTCCGCTAGCACCAAGGGACCTTCTGTGTTCCCTCT |
| | | GGCTCCTTCTTCTAAGTCCACTTCCGGTGGTACAGCAGCTCTG |
| | | GGTTGTCTGGTGAAGGATTACTTCCCAGAACCAGTGACTGTG |
| | | TCCTGGAACTCCGGAGCTCTGACTTCTGGAGTGCATACTTTC |
| | | CCAGCAGTGCTGCAATCTAGCGGACTGTACTCTCTGTCTTCC |
| | | GTGGTGACTGTGCCTTCTTCTTCCCTGGGGACTCAAACTTACA |
| | | TCTGCAACGTGAACCACAAGCCCTCCAACACCAAGGTGGAC |
| | | AAGAAGGTGGAGCCAAAGAGCTGCGATAAGACCCACACCTG |
| | | TCCACCTTGTCCAGCTCCAGAACTGCTGGGTGGGCCTTCTGT |
| | | GTTTCTGTTCCCACCTAAGCCAAAGGATACCCTGATGATCTC |
| | | TAGGACCCCAGAAGTGACCTGTGTGGTCGTCGATGTGTCTCA |
| | | TGAAGACCCTGAAGTGAAGTTCAACTGGTACGTGGACGGGG |
| | | TGGAAGTGCATAACGCAAAGACCAAGCCCAGGGAAGAGCAA |
| | | TACAACTCCACCTACAGGGTGGTCTCCGTCCTGACAGTCCTG |
| | | CATCAGGATTGGCTGAACGGCAAGGAGTACAAGTGCAAGGT |
| | | CTCCAATAAAGCCCTGCCTGCCCCTATCGAGAAAACCATTAG |
| | | CAAAGCCAAAGGCCAGCCCAGGGAGCCCCAGGTCTATACAC |
| | | TGCCCCCCAGCAGGGAGGAGATGACAAAAAATCAGGTCAGC |
| | | CTGACATGCCTGGTCAAAGGCTTTTATCCCAGCGACATTGCC |
| | | GTCGAGTGGGAGTCCAATGGCCAGCCCGAGAATAATTATAA |
| | | AACAACACCCCCCGTCCTGGACAGCGACGGCAGCTTTTTTCT |
| | | GTATAGCAAACTGACAGTCGATAAAAGCAGGTGGCAGCAGG |
| | | GCAATGTCTTTTCCTGCAGCGTCATGCACGAGGCCCTGCACA |
| | | ATCACTATACTCAGAAAAGCCTGAGCCTGTCCCCCGGGAAAT |
| | | GAGCGGCCGC |
| 49 | 11256H5- | GAATTCGCCGCCACCATGGTGCTGCAGACCCAGGTGTTCATC |
| | LC cDNA | TCCCTGCTGCTGTGGATCTCCGGCGCTTATGGCTCCTACGTGC |
| | | TGACCCAGCCTAGCAGCGTGTCCGTGTCCCCTGGCCAGACCG |
| | | CTAGGATCACCTGTAGCGGCGATGTGCTGGCCAAGAAGTAC |
| | | GCTCGGTGGTTCCAGCAGAAGCCTGGCCAGCCTCCTGTGCTG |
| | | CTGATCTATAAGGACACCGAGCGGCCTAGCGGCATCCCTGAG |
| | | AGGTTCTCCGGCTCCTCCTCCGGCACCACCGCTACCCTGACC |
| | | ATCTCCGGCGCCCAGGCTGAGGATGAGGCCGATTACTATTGC |
| | | TACAGCGCTGCCGACAATAAGGGCTTCTTCGGCACCGGCACC |
| | | AAGGTGACCGTGCTGCGTACGGTGGCTGCACCTTCTGTGTTC |
| | | ATCTTCCCTCCATCTGATGAGCAGCTGAAGTCTGGAACCGCA |
| | | TCTGTCGTCTGTCTGCTGAACAACTTTTACCCCAGGGAGGCT |
| | | AAGGTCCAATGGAAGGTGGACAACGCCCTGCAGTCTGGTAA |
| | | TAGCCAGGAAAGCGTGACCGAACAGGATTCCAAGGACTCCA |
| | | CCTACTCCCTGTCCTCCACACTGACACTGAGCAAAGCCGACT |
| | | ATGAAAAGCACAAAGTGTATGCCTGCGAGGTCACTCATCAG |
| | | GGCCTGTCCAGCCCCGTGACTAAAAGCTTTAATAGGGGGGA |
| | | GTGCTGAGCGGCCGC |

### LIST OF ENUMERATED EMBODIMENTS

Embodiment 1. An anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

Embodiment 2. The anti-syndecan-2 antibody of Embodiment 1, wherein the first amino acid sequence is selected from the group consisting of SEQ ID NOs: 1, 3, 5, 7, and 9 and the second amino acid sequence is selected from the group consisting of SEQ ID NOs: 2, 4, 6, 8, and 10.

Embodiment 3. The anti-syndecan-2 antibody of Embodiment 1 being an IgA, an IgD, an IgE, an IgG, or an IgM.

Embodiment 4. The anti-syndecan-2 antibody of Embodiment 1, further comprising a heavy chain constant region and a light chain constant region, wherein the heavy chain constant region comprises a third amino acid sequence having at least 90.5% sequence identity to SEQ ID NO: 36 and the light chain constant region comprises a fourth amino acid sequence having at least 91.5% sequence identity to SEQ ID NO: 37.

Embodiment 5. The anti-syndecan-2 antibody of Embodiment 4, wherein the third amino acid sequence is SEQ ID NO: 36 and the fourth amino acid sequence is SEQ ID NO: 37.

Embodiment 6. The anti-syndecan-2 antibody of Embodiment 5, wherein a fifth amino acid sequence comprising SEQ ID NO: 38 is fused to the heavy chain variable region and a sixth amino acid sequence comprising SEQ ID NO: 39 is fused to the light chain variable region.

Embodiment 7. The anti-syndecan-2 antibody of Embodiment 2, wherein the first amino acid sequence and the second amino acid sequence are respectively selected from the group consisting of SEQ ID NOs: 1 and 2, 3 and 4, 5 and 6, 7 and 8, and 9 and 10.

Embodiment 8. A method of treating a syndecan-2 associated disease in a subject in need thereof, the method comprising administering to the subject an effective amount of an anti-syndecan-2 antibody to treat the ARDS, the anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

Embodiment 9. The method of Embodiment 8, wherein the syndecan-2 associated disease is acute respiratory distress syndrome (ARDS), stroke, a neurological disease in which the blood brain barrier (BBB) is altered or disrupted, neovascular eye disease, a cardiovascular disease with a component of vascular hyperpermeability.

Embodiment 10. The method of Embodiment 9, wherein the syndecan-2 associated disease is coronavirus disease 2019-induced ARDS (COVID-19-induced ARDS).

Embodiment 11. The method of any one of Embodiments 8-10, wherein the anti-syndecan-2 antibody is conjugated to a heterologous peptide.

Embodiment 12. The method of any one of Embodiments 8-11, wherein the subject is a mammal.

Embodiment 13. The method of any one of Embodiments 8-12, wherein the subject is a human.

Embodiment 14. The method of any one of Embodiments 8-13, wherein the anti-syndecan-2 antibody is administered in a dosage form further comprising at least one pharmaceutically acceptable carrier.

Embodiment 15. A method of treating acute respiratory distress syndrome (ARDS) in a subject in need thereof, the method comprising administering to the subject an effective amount of a syndecan-2 disrupting peptide comprising an amino acid sequence set forth in SEQ ID NOs: 31, 32, or 33.

Embodiment 16. The method of Embodiment 15, wherein the ARDS is coronavirus disease 2019-induced ARDS (COVID-19-induced ARDS).

Embodiment 17. The method of Embodiment 15 or claim 16, wherein the syndecan-2 disrupting peptide is conjugated to a heterologous peptide.

Embodiment 18. The method of Embodiment 16, wherein the heterologous peptide is selected from the group consisting of a cell penetrating peptide, a secretion signal peptide, or a stability enhancing domain.

Embodiment 19. The method of any one of Embodiments 15-18, wherein the subject is a mammal.

Embodiment 20. The method of any one of Embodiments 15-19, wherein the subject is a human.

Embodiment 21. The method of any one of Embodiments 15-20, wherein the syndecan-2 disrupting peptide is administered in a dosage form further comprising at least one pharmaceutically acceptable carrier.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

2. The anti-syndecan-2 antibody of claim 1, wherein the first amino acid sequence is selected from the group consisting of SEQ ID NOs: 1, 3, 5, 7, and 9 and the second amino acid sequence is selected from the group consisting of SEQ ID NOs: 2, 4, 6, 8, and 10.

3. The anti-syndecan-2 antibody of claim 1 being an IgA, an IgD, an IgE, an IgG, or an IgM.

4. The anti-syndecan-2 antibody of claim 1, further comprising a heavy chain constant region and a light chain constant region, wherein the heavy chain constant region comprises a third amino acid sequence having at least 90.5% sequence identity to SEQ ID NO: 36 and the light chain constant region comprises a fourth amino acid sequence having at least 91.5% sequence identity to SEQ ID NO: 37.

5. The anti-syndecan-2 antibody of claim 4, wherein the third amino acid sequence is SEQ ID NO: 36 and the fourth amino acid sequence is SEQ ID NO: 37.

6. The anti-syndecan-2 antibody of claim 5, wherein a fifth amino acid sequence comprising SEQ ID NO: 38 is fused to the heavy chain variable region and a sixth amino acid sequence comprising SEQ ID NO: 39 is fused to the light chain variable region.

7. The anti-syndecan-2 antibody of claim 2, wherein the first amino acid sequence and the second amino acid sequence are respectively selected from the group consisting of SEQ ID NOs: 1 and 2, 3 and 4, 5 and 6, 7 and 8, and 9 and 10.

8. A method of treating a syndecan-2 associated disease in a subject in need thereof, the method comprising administering to the subject an effective amount of an anti-syndecan-2 antibody to treat the ARDS, the anti-syndecan-2 antibody comprising a heavy chain variable region and a light chain variable region, wherein the heavy chain variable region comprises a first amino acid sequence having at least 90.5% sequence identity to at least one selected from the group consisting of SEQ ID NOs: 1, 3, 5, 7, and 9 and the light chain variable region comprises a second amino acid sequence having at least 91.5% sequence identity to at least one selected from the group consisting of SEQ ID NOs: 2, 4, 6, 8, and 10.

9. The method of claim 8, wherein the syndecan-2 associated disease is acute respiratory distress syndrome (ARDS), stroke, a neurological disease in which the blood brain barrier (BBB) is altered or disrupted, neovascular eye disease, a cardiovascular disease with a component of vascular hyperpermeability.

10. The method of claim 9, wherein the syndecan-2 associated disease is coronavirus disease 2019-induced ARDS (COVID-19-induced ARDS).

11. The method of claim 8, wherein the anti-syndecan-2 antibody is conjugated to a heterologous peptide.

12. The method of claim 8, wherein the subject is a mammal.

13. The method of claim 8, wherein the subject is a human.

14. The method of claim 8, wherein the anti-syndecan-2 antibody is administered in a dosage form further comprising at least one pharmaceutically acceptable carrier.

15. A method of treating acute respiratory distress syndrome (ARDS) in a subject in need thereof, the method comprising administering to the subject an effective amount of a syndecan-2 disrupting peptide comprising an amino acid sequence set forth in SEQ ID NOs: 31, 32, or 33.

16. The method of claim 15, wherein the ARDS is coronavirus disease 2019-induced ARDS (COVID-19-induced ARDS).

17. The method of claim 15, wherein the syndecan-2 disrupting peptide is conjugated to a heterologous peptide.

18. The method of claim 16, wherein the heterologous peptide is selected from the group consisting of a cell penetrating peptide, a secretion signal peptide, or a stability enhancing domain.

19. The method of claim 15, wherein the subject is a mammal.

20. The method of claim 15, wherein the subject is a human.

21. The method of claim 15, wherein the syndecan-2 disrupting peptide is administered in a dosage form further comprising at least one pharmaceutically acceptable carrier.


SEQUENCE LISTING (51 sequences)

SEQ ID NO: 1 - protein, 118 aa, Artificial Sequence
  11526A8-Heavy Chain Variable Region
EVQLVQSGGGVVQPGRSLRLSCAASGFTFSRDGMHWVRQAPGKGLEWVAVIWNDGSNKYYADSVKGRFTISRDNSKNTLYLQMNSLRAEDTAVYYCARHYGDYGMDVWGQGTTVTVSS

SEQ ID NO: 2 - protein, 108 aa, Artificial Sequence
  11526A8- Light Chain Variable Region
SSELTQPSSVSMSPGQTARITCSGDVLAKKYARWFQQKPGQAPVLVIYKDSERPSGIPERFSGSSSGTTVTLTISGVQAEDEADYYCQSADSSGTRWVFGGGTKLTVL

SEQ ID NO: 3 - protein, 123 aa, Artificial Sequence
  11526B9-Heavy Chain Variable Region
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPRGIRAYGGYSLDYWGQGTLVTVSS

SEQ ID NO: 4 - protein, 110 aa, Artificial Sequence
  11526B9-Light Chain Variable Region
QTVVTQEPSFSVSPGGTVTLTCGLNSGSVSSRFFPSWYQQTPGQPPRTLIYSTRIRSSGVPDRFSGFILGDKAALTITGAQADDESVYYCLLYMGSGIWVFGGGTKLTVL

SEQ ID NO: 5 - protein, 122 aa, Artificial Sequence
  11526E1-Heavy Chain Variable Region
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKHIRGQRYFAYMDVWGKGTLVTVSS

SEQ ID NO: 6 - protein, 107 aa, Artificial Sequence
  11526E1-Light Chain Variable Region
AIRMTQSPSSVSASVGDRVTITCRASQGISSRLAWYQQKPGKAPKLLIYAASSLQSGVPSRFSGSGSGTDFTLTISSVQPEDFATYYCQQANSFPLTFGGGTKVDIK

SEQ ID NO: 7 - protein, 117 aa, Artificial Sequence
  11526F3-Heavy Chain Variable Region
QVQLQQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRRGGGDYWGQGTLVTVSS

SEQ ID NO: 8 - protein, 110 aa, Artificial Sequence
  11526F3-Light Chain Variable Region
QAVVTQEPSFSVSPGGTVTLTCGLSSGSVSTTYYPSWYQLTPGQAPRTLIYNTNTRSSGVPDRFSGSILGNKAALTITGAQADDESYYYCLLYMGRGVRVFGGGTKLTVL

SEQ ID NO: 9 - protein, 119 aa, Artificial Sequence
  11526H5-Heavy Chain Variable Region
QVQLVESGGGVVQPGRSLRLSCAASGFTFSSYAVHWVRQAPGKGLEWVAVISYDGSNKYYADSVKGRFTISRDNSKNTLFLQMNSLRAEDTAVYYCARRGLRGYAFDIWGQGTLVTVSS

SEQ ID NO: 10 - protein, 106 aa, Artificial Sequence
  11526H5-Light Chain Variable Region
SYVLTQPSSVSVSPGQTARITCSGDVLAKKYARWFQQKPGQPPVLLIYKDTERPSGIPERFSGSSSGTTATLTISGAQAEDEADYYCYSAADNKGFFGTGTKVTVL

SEQ ID NO: 11 - protein, 448 aa, Artificial Sequence
  11526A8-Heavy Chain
EVQLVQSGGGVVQPGRSLRLSCAASGFTFSRDGMHWVRQAPGKGLEWVAVIWNDGSNKYYADSVKGRFTISRDNSKNTLYLQMNSLRAEDTAVYYCARHYGDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  11526A8- Light Chain
SSELTQPSSVSMSPGQTARITCSGDVLAKKYARWFQQKPGQAPVLVIYKDSERPSGIPERFSGSSSGTTVTLTISGVQAEDEADYYCQSADSSGTRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 453 aa, Artificial Sequence
  11526B9-Heavy Chain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPRGIRAYGGYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Artificial Sequence
  11526B9-Light Chain
QTVVTQEPSFSVSPGGTVTLTCGLNSGSVSSRFFPSWYQQTPGQPPRTLIYSTRIRSSGVPDRFSGFILGDKAALTITGAQADDESVYYCLLYMGSGIWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 452 aa, Artificial Sequence
  11526E1-Heavy Chain
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKHIRGQRYFAYMDVWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 214 aa, Artificial Sequence
  11526E1-Light Chain
AIRMTQSPSSVSASVGDRVTITCRASQGISSRLAWYQQKPGKAPKLLIYAASSLQSGVPSRFSGSGSGTDFTLTISSVQPEDFATYYCQQANSFPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 447 aa, Artificial Sequence
  11526F3-Heavy Chain
QVQLQQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRRGGGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 217 aa, Artificial Sequence
  11526F3-Light Chain
QAVVTQEPSFSVSPGGTVTLTCGLSSGSVSTTYYPSWYQLTPGQAPRTLIYNTNTRSSGVPDRFSGSILGNKAALTITGAQADDESYYYCLLYMGRGVRVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Artificial Sequence
  11526H5-Heavy Chain
QVQLVESGGGVVQPGRSLRLSCAASGFTFSSYAVHWVRQAPGKGLEWVAVISYDGSNKYYADSVKGRFTISRDNSKNTLFLQMNSLRAEDTAVYYCARRGLRGYAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 213 aa, Artificial Sequence
  11526H5-Light Chain
SYVLTQPSSVSVSPGQTARITCSGDVLAKKYARWFQQKPGQPPVLLIYKDTERPSGIPERFSGSSSGTTATLTISGAQAEDEADYYCYSAADNKGFFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 467 aa, Artificial Sequence
  11526A8-Heavy Chain- with tag
MKHLWFFLLLVAAPRWVLSEVQLVQSGGGVVQPGRSLRLSCAASGFTFSRDGMHWVRQAPGKGLEWVAVIWNDGSNKYYADSVKGRFTISRDNSKNTLYLQMNSLRAEDTAVYYCARHYGDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 235 aa, Artificial Sequence
  11526A8- Light Chain- with tag
MVLQTQVFISLLLWISGAYGSSELTQPSSVSMSPGQTARITCSGDVLAKKYARWFQQKPGQAPVLVIYKDSERPSGIPERFSGSSSGTTVTLTISGVQAEDEADYYCQSADSSGTRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 472 aa, Artificial Sequence
  11526B9-Heavy Chain- with tag
MKHLWFFLLLVAAPRWVLSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPRGIRAYGGYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 237 aa, Artificial Sequence
  11526B9-Light Chain- with tag
MVLQTQVFISLLLWISGAYGQTVVTQEPSFSVSPGGTVTLTCGLNSGSVSSRFFPSWYQQTPGQPPRTLIYSTRIRSSGVPDRFSGFILGDKAALTITGAQADDESVYYCLLYMGSGIWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 471 aa, Artificial Sequence
  11526E1-Heavy Chain- with tag
MKHLWFFLLLVAAPRWVLSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKHIRGQRYFAYMDVWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 234 aa, Artificial Sequence
  11526E1-Light Chain- with tag
MVLQTQVFISLLLWISGAYGAIRMTQSPSSVSASVGDRVTITCRASQGISSRLAWYQQKPGKAPKLLIYAASSLQSGVPSRFSGSGSGTDFTLTISSVQPEDFATYYCQQANSFPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 466 aa, Artificial Sequence
  11526F3-Heavy Chain- with tag
MKHLWFFLLLVAAPRWVLSQVQLQQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRRGGGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 237 aa, Artificial Sequence
  11526F3-Light Chain- with tag
MVLQTQVFISLLLWISGAYGQAVVTQEPSFSVSPGGTVTLTCGLSSGSVSTTYYPSWYQLTPGQAPRTLIYNTNTRSSGVPDRFSGSILGNKAALTITGAQADDESYYYCLLYMGRGVRVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 468 aa, Artificial Sequence
  11526H5-Heavy Chain- with tag
MKHLWFFLLLVAAPRWVLSQVQLVESGGGVVQPGRSLRLSCAASGFTFSSYAVHWVRQAPGKGLEWVAVISYDGSNKYYADSVKGRFTISRDNSKNTLFLQMNSLRAEDTAVYYCARRGLRGYAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 233 aa, Artificial Sequence
  11526H5-Light Chain- with tag
MVLQTQVFISLLLWISGAYGSYVLTQPSSVSVSPGQTARITCSGDVLAKKYARWFQQKPGQPPVLLIYKDTERPSGIPERFSGSSSGTTATLTISGAQAEDEADYYCYSAADNKGFFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 18 aa, Artificial Sequence
  Syndecan-2 disrupting peptide human
PAEEDTNVYTEKHSDSLF

SEQ ID NO: 32 - protein, 18 aa, Artificial Sequence
  Syndecan-2 disrupting peptide mouse
PAIKSTDVYTEKHSDNLF

SEQ ID NO: 33 - protein, 137 aa, Artificial Sequence
  SDC2 extracellular
MYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILL

SEQ ID NO: 34 - protein, 201 aa, Artificial Sequence
  SDC2 Human
MRRAWILLTLGLVACVSAESRAELTSDKDMYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILLLVYRMRKKDEGSYDLGERKPSSAAYQKAPTKEFYA

SEQ ID NO: 35 - protein, 202 aa, Artificial Sequence
  SDC2 Mouse
MQRAWILLTLGLMACVSAETRTELTSDKDMYLDNSSIEEASGVYPIDDDDYSSASGSGADEDIESPVLTTSQLIPRIPLTSAASPKVETMTLKTQSITPAQTESPEETDKEEVDISEAEEKLGPAIKSTDVYTEKHSDNLFKRTEVLAAVIAGGVIGFLFAIFLILLLVYRMRKKDEGSYDLGERKPSSAAYQKAPTKEFYA

SEQ ID NO: 36 - protein, 330 aa, Artificial Sequence
  Anti-Syndecan 2 IgG heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 107 aa, Artificial Sequence [sequence omitted]

SEQ ID NO: 38 - protein, 18 aa, Artificial Sequence
  Anti-Syndecan 2 heavy chain tag
MKHLWFFLLLVAAPRWVL

SEQ ID NO: 39 - protein, 19 aa, Artificial Sequence
  Anti-Syndecan 2 light chain tag
MVLQTQVFISLLLWISGAY

SEQ ID NO: 40 - DNA, 1427 bp, Artificial Sequence
  11256A8-HC cDNA
gaattcgccgccaccatgaagcacctgtggttcttcctgctgctggtggccgccccccggtgggtgctgtccgaggtgcagctggtgcagtccggcggcggagtggtgcagcctggaaggagcctgaggctgtcctgcgctgcttctggcttcaccttcagccgggacggcatgcactgggtgaggcaggcccccggaaagggcctggagtgggtggctgtgatctggaatgacggctccaacaagtactacgccgattccgtgaagggccggttcaccatcagccgggacaattccaagaataccctgtacctgcagatgaatagcctgcgggccgaggataccgctgtgtattactgtgctcggcactatggcgactacggcatggacgtgtggggccagggcaccaccgtgaccgtgagctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 41 - DNA, 731 bp, Artificial Sequence
  11256A8-LC cDNA
gaattcgccgccaccatggtgctgcagacccaggtgttcatctccctgctgctgtggatcagcggcgcctacggctcctccgagctgacccagcctagctccgtgagcatgtcccctggccagaccgccaggatcacctgctccggcgatgtgctggctaagaagtacgctaggtggttccagcagaagcccggccaggctcccgtgctggtgatctacaaggatagcgagcggccctccggcatccctgagaggttcagcggcagcagcagcggcaccaccgtgaccctgaccatctccggcgtgcaggccgaggacgaggctgactactattgccagtccgctgatagctccggcaccaggtgggtgttcggcggcggaaccaagctgaccgtgctgcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 42 - DNA, 1441 bp, Artificial Sequence
  11256B9-HC cDNA
gaattcgccgccaccatgaagcacctgtggttcttcctgctgctggtggctgctccccggtgggtgctgagccaggtgcagctggtgcagagcggcgctgaggtgaagaagcctggcgcttctgtgaaggtgtcctgtaaggccagcggctataccttcacctcctactacatgcactgggtgaggcaggctcctggccagggcctggagtggatgggcatcatcaatcccagcggcggctccacctcctacgctcagaagttccagggccgggtgaccatgaccagggacacctccaccagcaccgtgtacatggagctgtcctccctgcggagcgaggataccgccgtgtactattgtgcccggcctaggggcatcagggcctacggaggctacagcctggattattggggccagggcaccctggtgaccgtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggttggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 43 - DNA, 737 bp, Artificial Sequence
  11256B9-LC cDNA
gaattcgccgccaccatggtgctgcagacccaggtgttcatctccctgctgctgtggatctccggcgcctatggccagaccgtggtgacccaggagcctagcttctccgtgtcccctggcggcaccgtgaccctgacctgcggactgaacagcggcagcgtgtccagcaggttcttccctagctggtaccagcagacccccggccagcccccaagaaccctgatctatagcaccaggatccggagcagcggcgtgcctgacaggttcagcggcttcatcctgggcgacaaggccgctctgaccatcaccggcgcccaggctgacgatgagagcgtgtattactgtctgctgtacatgggctccggcatctgggtgttcggcggcggaaccaagctgaccgtgctgcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 44 - DNA, 1439 bp, Artificial Sequence
  11256E1-HC cDNA
gaattcgccgccaccatgaagcacctgtggttcttcctgctgctggtggctgccccccggtgggttctgtcccaggtgcagctggtgcagtccggcggcggactggtgcagcctggaggatccctgaggctgagctgcgctgccagcggcttcaccttcagctcctacgctatgtcctgggtgcggcaggcccccggaaagggactggagtgggtgagcgccatctccggcagcggcggaagcacctactacgccgactccgtgaagggcaggttcaccatctccagggataactccaagaacaccctgtatctgcagatgaacagcctgcgggctgaggataccgccgtgtattactgcgccaagcacatccggggccagaggtacttcgcctacatggatgtgtggggcaagggcaccctggtgaccgtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 45 - DNA, 728 bp, Artificial Sequence
  11256E1-LC cDNA
gaattcgccgccaccatggtgctgcagacccaggtgttcatcagcctgctgctgtggatctccggcgcttatggcgctatccggatgacccagagccccagctccgtgtccgctagcgtgggcgacagggtgaccatcacctgtagggcctcccagggcatctcctcccggctggcttggtatcagcagaagcccggcaaggctcccaagctgctgatctatgctgccagctccctgcagagcggcgtgccttcccggttcagcggctccggatccggcaccgacttcaccctgaccatctccagcgtgcagcctgaggacttcgctacctactattgtcagcaggccaattccttccctctgaccttcggcggcggcaccaaggtggacatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 46 - DNA, 1424 bp, Artificial Sequence
  11256F3-HC cDNA
gaattcgccgccaccatgaagcacctgtggttcttcctgctgctggtggctgcccctaggtgggtgctgagccaggtgcagctgcagcagagcggcgctgaggtgaagaagcccggcgccagcgtgaaggtgagctgtaaggcttccggctacaccttcacctcctactacatgcactgggtgaggcaggctcccggccagggactggagtggatgggcatcatcaaccctagcggcggctccacctcctacgcccagaagttccagggcagggtgaccatgaccagggacacctccaccagcaccgtgtacatggagctgtccagcctgaggagcgaggataccgctgtgtattactgtgctcgggaccggcggggcggcggagactactggggacagggaaccctggtgaccgtgagctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 47 - DNA, 737 bp, Artificial Sequence
  11256F3-LC cDNA
gaattcgccgccaccatggtgctgcagacccaggtgttcatctccctgctgctgtggatctccggcgcttatggccaggctgtggtgacccaggagccttccttcagcgtgtcccctggcggcaccgtgaccctgacctgtggcctgtccagcggcagcgtgagcaccacctactacccttcctggtaccagctgacccctggccaggcccctcggacactgatctataataccaacacccggagcagcggcgtgcccgatcggttcagcggctccatcctgggcaataaggctgctctgaccatcaccggcgctcaggctgacgatgagagctattactactgtctgctgtacatgggcaggggcgtgcgggtgttcggcggaggaaccaagctgaccgtgctgcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 48 - DNA, 1430 bp, Artificial Sequence
  11256H5-HC cDNA
gaattcgccgccaccatgaagcacctgtggttcttcctgctgctggtggctgcccctcggtgggtgctgtcccaggtgcagctggtggagagcggcggcggagtggtgcagccaggaagaagcctgaggctgagctgtgccgcttccggcttcaccttcagctcctacgctgtgcactgggtgcggcaggctcccggaaagggcctggagtgggtggctgtgatctcctacgatggctccaacaagtactatgccgactccgtgaagggccggttcaccatctcccgggataattccaagaacaccctgttcctgcagatgaatagcctgagggccgaggacaccgctgtgtactattgcgcccggaggggcctgcggggctatgccttcgatatctggggccagggcaccctggtgaccgtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 49 - DNA, 725 bp, Artificial Sequence
  11256H5-LC cDNA
gaattcgccgccaccatggtgctgcagacccaggtgttcatctccctgctgctgtggatctccggcgcttatggctcctacgtgctgacccagcctagcagcgtgtccgtgtcccctggccagaccgctaggatcacctgtagcggcgatgtgctggccaagaagtacgctcggtggttccagcagaagcctggccagcctcctgtgctgctgatctataaggacaccgagcggcctagcggcatccctgagaggttctccggctcctcctccggcaccaccgctaccctgaccatctccggcgcccaggctgaggatgaggccgattactattgctacagcgctgccgacaataagggcttcttcggcaccggcaccaaggtgaccgtgctgcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 50 - protein, 68 aa, Artificial Sequence
  SDC2 Human
LPKILLTSAAPKVETTTLNIQNKIPAQTKSPEETDKEKVHLSDSERKMDPAEEDTNVYTEKHSDSLFK

SEQ ID NO: 51 - protein, 69 aa, Artificial Sequence
  SDC2 Mouse
IPRIPLTSAASPKVETMTLKTQSITPAQTESPEETDKEEVDISEAEEKLGPAIKSTDVYTEKHSDNLFK